(19) United States
(12) Patent Application Publication
(10) Pub. No.: US 2026/0027268 A1
(43) Pub. Date: Jan. 29, 2026

(54) CATHETERS

(71) Applicant: ConvaTec Limited, Flintshire (GB)

(72) Inventors: David Pollard, Merseyside (GB); Rachel Pytel, Newton, MA (US); Luca Barbieri, Chester (GB)

(21) Appl. No.: 19/343,068

(22) Filed: Sep. 29, 2025

Related U.S. Application Data
(63) Continuation of application No. PCT/GB2024/050807, filed on Mar. 26, 2024.
(60) Provisional application No. 63/455,330, filed on Mar. 29, 2023.

(30) Foreign Application Priority Data
May 11, 2023 (GB) 2306937.0

(51) Int. Cl. A61L 29/08; A61L 29/14; A61M 25/00

(52) U.S. Cl. CPC A61L 29/085; A61L 29/145; A61M 25/0017; A61M 25/002; A61M 25/0045

(57) ABSTRACT

The present disclosure is a urinary catheter that has a hollow polymeric tubular body that has a base polymer and an amphiphilic additive. In one aspect of the disclosure, at least a part of the body is coated with an aqueous gel, wherein the gel comprises water in a total amount of between 5-99 wt. %.

### TECHNICAL FIELD OF THE INVENTION

The present invention relates to catheters coated with aqueous gels, as well as to aqueous gel lubricating and/or wetting agents.

### BACKGROUND TO THE INVENTION

Urinary catheterisation is a process involving insertion of a catheter through an individual's urethra and into their bladder, where it is retained to empty the bladder of urine. There are two major types of urinary catheterisation—intermittent catheterisation and long-term catheterisation. Intermittent urinary catheterisation involves retaining the catheter in the bladder for only the time period required for emptying, after which the catheter is removed. The process differs from long-term catheterisation, which makes use of an indwelling or Foley catheter that is inserted into the bladder for long periods of time (several days to months) to discharge the residual urine of the bladder continuously throughout the day.

Catheterisation is often used by patients suffering from abnormalities of the urinary system, resulting in urinary incontinence and/or a lack of control in permitting voluntary urination. Such individuals would typically make use of intermittent catheters several times a day.

Catheters are useful devices, providing users with independence and freedom to self-catheterise as and when required, without having to rely on trained personnel to be present. This, however, increases the need for catheters to be user friendly: in particular, both easy to insert and remove with minimum discomfort caused, and safe to use with features for minimising risk of infection. Users often report experiencing pain and discomfort upon insertion and/or removal of catheters. Users have, for instance, reported experiencing bladder spasms, burning sensations, and bleeding.

Surface coatings and additives for catheters have been used to help in alleviating these issues. U.S. Pat. No. 10,058,638 B2 and U.S. Pat. No. 9,186,438 B2 describe the use of a catheter containing a polymer mixture of a thermoplastic or thermocuring polymer base material and an amphiphilic block copolymer lubricious additive. The amphiphilic block copolymer contains both a hydrophobic and hydrophilic portion. The hydrophilic portion diffuses to the surface of the catheter due to incompatibility with the hydrophobic base material and provides for a lubricious surface coating. Interactions between the hydrophobic portion of the amphiphilic molecule and the base material assist in reducing migration of the amphiphilic molecule from the catheter.

Catheters comprising such additives must typically be kept well lubricated to activate and/or maintain catheter surface lubricity. Conventionally, catheters have been lubricated using water or aqueous solutions. Catheters may be packaged wet in direct contact with water or an aqueous solution, or may require that the user wets the catheter prior to use.

However, such methods have several drawbacks. For instance, when stored wet, the additive has been known to migrate out of the catheter and into the water or solution, reducing its effectiveness. Additionally, wetting a catheter before use can be challenging for users, particularly for untrained individuals practicing self-catheterisation. Liquid wetting agents can also be drippy and messy, making the process of wetting a catheter even more unpleasant for the user.

Furthermore, repeated lubrication may be required, and catheters can often suffer from dry-out, becoming rough and sticky, and potentially leading to additive delamination. Catheters have also been known to swell when wetted, further complicating the lubrication process. The logistics of storing catheters with liquid wetting agents can also present issues. Often, catheter packaging needs to be modified to accommodate a liquid wetting agent without it leaking out, which can add to the overall cost and complexity of manufacture and storage of the catheter.

There is therefore a need for alternative approaches to achieve catheter lubrication that can overcome or ameliorate limitations of current methods. It is an aim of embodiments of the present invention to overcome and/or ameliorate at least one problem of the prior art above.

It is also an aim of embodiments of the present invention to overcome or mitigate at least one problem of the prior art, whether expressly described herein or not.

### SUMMARY OF THE INVENTION

According to a first aspect of the invention, there is provided a urinary catheter comprising a hollow polymeric tubular body comprising a base polymer and an amphiphilic additive, wherein at least a part of the body is coated with an aqueous gel.

According to a second aspect of the invention, there is provided a urinary catheter comprising a hollow polymeric tubular body comprising a base polymer and an amphiphilic additive, wherein at least part of the body is coated with an aqueous gel, wherein the gel comprises water in a total amount of between 5-99 wt. %.

Coating such a catheter comprising an amphiphilic additive with an aqueous gel has been found to result in a catheter with excellent lubricity. Surprisingly, the gel coating provides for a catheter body with a very low coefficient of friction that is in line with coefficients achieved directly after lubrication with water or an aqueous solution. However, the use of a gel provides a number of further advantages over use of a liquid, such as water or an aqueous solution. Advantages include:
[symbol] The aqueous gel provides a coating that adheres better to the body of the catheter and provides long-lasting lubrication, in comparison with liquids which can easily drip or run off.
[symbol] The aqueous gel is easy to handle and can be easily applied to the body of the catheter.
[symbol] The catheter can be more easily stored with the aqueous gel coating—e.g. in a packaging container, compared to liquids which can be difficult to store and contain.
[symbol] The aqueous gel also provides a reduced risk of contamination compared to liquids which can become easily contaminated.
[symbol] The aqueous gel can allow for low levels of amphiphilic additive migration out of the catheter and into the gel.

The following statements may be applied mutatis mutandis to the first and second aspects of the invention.

The definition of a gel includes: a nonfluid colloidal network or polymer network that is expanded throughout its whole volume by a fluid; and a porous three-dimensional semi-solid network that expands in a stable fashion.

In preferred embodiments, the catheter is an intermittent urinary catheter. Such a catheter is typically inserted into a body for short time periods, such as less than a day. Alternatively, the catheter may be an indwelling (Foley) catheter. Such a catheter is typically inserted and kept in a body for long periods of time, such as several days to months. The catheter may be a single-use or multiple-use catheter.

The amphiphilic additive comprises a hydrophobic portion and a hydrophilic portion. In cases where the base polymer is hydrophobic or generally hydrophobic, such as a polyolefin, the amphiphilic additive will diffuse towards and to an outer surface of the catheter body due to incompatibility of the hydrophilic portion of the amphiphilic additive with the hydrophobic base polymer.

The amphiphilic additive may be an amphiphilic lubricious additive.

Preferably, the amphiphilic additive is activated upon contact with the aqueous gel. The gel may encourage hydrophilic portions of amphiphilic additive molecules within the catheter to seek towards an outer surface of the catheter, which further enhances the lubricating effect of the additive.

In some embodiments, at least one amphiphilic additive is polymeric or oligomeric.

At least one amphiphilic additive may be an A-B block copolymer comprising a hydrophobic hydrocarbon A-block and a hydrophilic B-block. In some embodiments, one or both of the hydrophobic hydrocarbon A-block and the hydrophilic B-block may be branched. The hydrophobic A-block may comprise hydrophobic hydrocarbon chains branching therefrom. The hydrophobic hydrocarbon chains may be of shorter chain lengths than the hydrophobic hydrocarbon A-block. The hydrophilic B-block may comprise further hydrophilic B-blocks branching therefrom.

In some embodiments, the amphiphilic additive is a B-A-B tri-block copolymer comprising a hydrophobic hydrocarbon A-block and hydrophilic B-blocks.

In other embodiments, the amphiphilic additive is a graft copolymer. The graft copolymer may comprise a hydrophobic hydrocarbon A-block with hydrophilic B-blocks branching therefrom.

Alternatively, the graft copolymer may comprise a hydrophilic portion with hydrophobic portions branching therefrom.

In further embodiments, the amphiphilic additive is a brush copolymer. The additive may comprise a single hydrophilic B-block with more than one hydrophobic A-block branching from an end thereof. Alternatively, the additive may comprise a single hydrophobic A-block with more than one hydrophilic B-block branching from an end thereof. In the respective embodiments, the B-block or A-block may comprise 2, 3, 4, 5, 6, 7, 8, 9, 10, or more hydrophobic A-blocks or hydrophilic B-blocks branching from the end thereof.

In further embodiments, the amphiphilic additive is a star-block or multi-block copolymer comprising hydrophilic and hydrophobic monomer units.

In preferred embodiments, the amphiphilic additive is an A-B block copolymer comprising a hydrophobic A-block and a hydrophilic B-block.

Statements of invention below relating to the amphiphilic additive or a part thereof may be applied mutatis mutandis to each of the copolymer forms above.

In some embodiments, the B-block is a hydrophilic oligomer comprising at least 1, 2, 3, 4, or at least 5 monomer units. In some embodiments, the B-block comprises no greater than 20, 19, 18, 17, 16, 15, 14, 13, 12, 11, 10, 9, 8, 7, or no greater than 6 monomer units. In some embodiments, the B-block comprises between 2 and 15 monomer units, preferably between 2 and 10 monomer units. At least one monomer unit may be independently chosen from: alkylene oxides, alkylene glycols, epihalohydrins, unsaturated carboxylic acids, alkylene imines, lactones, vinyl alcohol, and vinyl alkanoates. At least one monomer unit may be independently selected from the group consisting of: alkylene oxides, alkylene glycols, epihalohydrins, unsaturated carboxylic acids, alkylene imines, lactones, vinyl alcohol, and vinyl alkanoates. At least one monomer unit may be preferably independently chosen from: ethylene oxide, propylene oxide, ethylene glycol, propylene glycol, epichlorohydrin, acrylic acid, methacrylic acid, ethylene imine, caprolactone, vinyl alcohol, and vinyl acetate. At least one monomer unit may be preferably independently selected from the group consisting of: ethylene oxide, propylene oxide, ethylene glycol, propylene glycol, epichlorohydrin, acrylic acid, methacrylic acid, ethylene imine, caprolactone, vinyl alcohol, and vinyl acetate. In some embodiments, at least one monomer unit comprises alkylene oxide groups independently selected from ethylene oxide and propylene oxide, and in preferred embodiments, all of the monomer units are ethylene oxide or all of the monomer units are propylene oxide.

The hydrophobic A-block may comprise a carbon chain of at least 5 carbon atoms, or at least 10, 15, 20, 25, 30, 35, or 40 carbon atoms. The hydrophobic portion may preferably comprise a carbon chain of between 20-52 carbon atoms.

In some embodiments, the A-block comprises a hydrocarbon chain block of the formula CH₃CH₂(CH₂CH₂)ₐ. The value of “a” may be between 5-25; for instance, “a” may be 5, 6, 7, 8, 9, 10, 11, 12, 13, 14, 15, 16, 17, 18, 19, 20, 21, 22, 23, 24, or 25, or a half integer of any of the above values. The value of “a” may preferably be between 9-25; for instance, “a” may be 9, 10, 11, 12, 13, 14, 15, 16, 17, 18, 19, 20, 21, 22, 23, 24, or 25, or a half integer of any of the above values.

In some embodiments, the amphiphilic additive is homogenously distributed with the base polymer. The amphiphilic additive may be uniformly distributed throughout the base polymer of the catheter body.

At least some of the amphiphilic additive may be at or on an outer surface of the body. By “at an outer surface”, it is meant that at least a portion of the additive forms part of the surface or protrudes from the surface. In some embodiments, part of the additive is retained or anchored in the body while part of the additive forms part of or protrudes from the outer surface of the body. At least part of the hydrophilic portion of the additive may protrude from or form part of the outer surface of the body, while at least part of the hydrophobic portion may be retained or anchored within the body.

The outer surface may comprise at least one member chosen from: the external-facing surface of the body, the lumen of the body and any eyelets present on the body. The outer surface may comprise at least one member of the group consisting of: the external-facing surface of the body, the lumen of the body and any eyelets present on the body. In preferred embodiments the outer surface is the external-facing surface of the body and/or the inner lumen. In some embodiments, the outer surface may comprise the external-facing surface of the body of the catheter, the inner lumen, and the eyelets.

The amphiphilic additive may be concentrated at or on the outer surface of the body. For example, at least 25%, 30%, 35%, 40%, 45%, 50%, 55%, 60%, 65%, 70%, 75%, 80%, 85%, 90%, or at least 95% of the number of molecules of the additive may be at or on the outer surface of the body.

In some embodiments, at least 25%, 30%, 35%, 40%, 45%, 50%, 55%, 60%, 65%, 70%, 75%, 80%, 85%, 90%, or at least 95% of the number of molecules of additive may have hydrophilic portions that are at or on the outer surface of the body.

In some embodiments, the additive is located at and/or on at least 50, 55, 60, 65, 70, 75, 80, 85, 90, 95, 96, 97, 98 or at least 99% of the outer surface area of the polymeric tubular body, preferably at least 75% or at least 90% of the outer surface area of the polymeric tubular body or between 75% and 100% of the outer surface area.

In some embodiments, the additive is present at a concentration of at least 0.1, 0.2, 0.3. 0.4. 0.5, 0.75, 1, 2, 3, 4, 5, 10, 15 or at least 20% by weight of the combination of base polymer and additive. The additive may be present a concentration of between 0.1-20%, and more preferably between 0.5-15% or 0.5-5% by weight of the combination of base polymer and additive.

In some embodiments, the additive comprises a layer that is on or that comprises a surface of the body, preferably the outer surface.

The layer comprising the additive may be on the surface of the body. In some embodiments, the layer comprising the additive is substantially separate from the body and the layer may be bonded to the body. The layer may be bonded to the body via covalent bonds, ionic bonds, hydrogen bonds, or Van der Waals forces. The additive may be bonded to the body via one or more surface linker groups which may be present on the additive, the body of the catheter or both.

In some embodiments, the layer comprising the additive may comprise the surface of the body. In such embodiments the layer may form the surface of the body. The layer may comprise a co-extruded layer which is melded with or is physically entangled with the body, and this may form an integral layer. The layer of additive may be integrally formed with the body.

In some embodiments, polymer diffusion occurs between the layer comprising the additive and the catheter body. The layer and the body may be held together by polymer chains extending across the interface between the layer and body. In some embodiments, the additive infiltrates the catheter body.

In some embodiments, the layer comprising the additive comprises or is on an inner surface of the body, an outer surface of the body, or both. The inner surface of the body may comprise a lumen of the catheter. In preferred embodiments, the layer comprising the additive comprises or is on at least an outer surface of the body.

In some embodiments, the layer comprising the additive is on or comprises at least 50, 55, 60, 65, 70, 75, 80, 85, 90, 95, 96, 97, 98 or at least 99% of the or each surface area of the body, preferably at least 75% or at least 90% of the or each surface area or between 75% and 100% of the or each surface area. In embodiments in which the layer comprising the additive comprises or is on both an inner and outer surface of the body, the additive may comprise at least 50, 55, 60, 65, 70, 75, 80, 85, 90, 95, 96, 97, 98 or at least 99% of each surface area of the body, preferably at least 75% or at least 90% of each surface area or between 75% and 100% of each surface area of both surfaces.

In some embodiments, at least 75% of the layer comprising the additive, or at least 76, 77, 78, 79, 80, 81, 82, 83, 84, 85, 86, 87, 88, 89, 90, 91, 92, 93, 94, 95, 96, 97, 98, 99, or 100% of the layer is the additive.

In some embodiments, the layer comprising the additive has an additive concentration of at least 0.1, 0.2, 0.3. 0.4. 0.5, 0.75, 1, 2, 3, 4, 5, 10, 15 or at least 20% by weight of the combination of base polymer and additive.

In some embodiments, the layer comprising the additive has an additive concentration of no greater than 70, 65, 60, 65, 60, 55, or of no greater than 50% by weight of the combination of the base polymer and additive.

The layer comprising the additive may have an additive concentration of greater than 5% by weight of the combination of the base polymer and additive. The layer may have an additive concentration of between 6-50% by weight of the combination of the base polymer and additive.

The layer comprising the additive may have an additive concentration of between 10-50% by weight of the combination of the base polymer and additive, or of between 15-50, 20-50, 25-50, 30-50, 35-50, 40-50, or of between 45-50% by weight of the combination of the base polymer and additive.

The layer comprising the additive may have an additive concentration of between 6-45% by weight of the combination of the base polymer and additive, or of between 6-40, 6-35, 6-30, 6-25, 6-20, 6-15, or of between 6-10% by weight of the combination of the base polymer and additive.

The layer comprising the additive may have an additive concentration of between 10-45% by weight of the combination of the base polymer and additive, or of between 15-45, 20-45, 25-45, 30-45, 35-45, 40-45, 10-40, 15-40, 20-40, 25-40, 30-40, 35-40, 10-35, 15-35, 20-35, 25-35, 30-35, 10-30, 15-30, 20-30, 25-30, 10-25, 15-25, 20-25, 10-20, 15-20, or of between 10-15% by weight of the combination of the base polymer and additive.

In some embodiments, the layer comprising the additive has a thickness of at least 1 μm, or of at least 2, 3, 4, 5, 6, 7, 8, 9, 10, 15, 20, 25, 30, 35, 40, 45, or of at least 50 μm.

In some embodiments, the layer comprising the additive has a thickness of no more than 10000 μm, or of no more than 9000, 8000, 7000, 6000, 5000, 4000, 3000, 2000, 1000, 900, 800, 700, 600, 500, 400, or of no more than 300 μm.

In some embodiments, the layer comprising the additive has a thickness of between 50-300 μm.

The layer comprising the additive may have a thickness of between 60-300 μm, or of between 80-300, 100-300, 120-300, 140-300, 160-300, 180-300, 200-300, 220-300, 240-300, 260-300, or of between 280-300 μm.

The layer comprising the additive may have a thickness of between 50-280 μm, or of between 50-260, 50-240, 50-220, 50-200, 50-180, 50-160, 50-140, 50-120, 50-100, 50-80, or of between 50-60 μm.

The layer comprising the additive may have a thickness of between 60-280 μm, or of between 80-280, 100-280, 120-280, 140-280, 160-280, 180-280, 200-280, 220-280, 240-280, 260-280, 60-260, 80-260, 100-260, 120-260, 140-260, 160-260, 180-260, 200-260, 220-260, 240-260, 60-240, 80-240, 100-240, 120-240, 140-240, 160-240, 180-240, 200-240, 220-240, 60-220, 80-220, 100-220, 120-220, 140-220, 160-220, 180-220, 200-220, 60-200, 80-200, 100-200, 120-200, 140-200, 160-200, 180-200, 60-180, 80-180, 100-180, 120-180, 140-180, 160-180, 60-160, 80-160, 100-160, 120-160, 140-160, 60-140, 80-140, 100-140, 120-140, 60-120, 80-120, 100-120, 60-100, 80-100, or of between 60-80 μm.

In preferred embodiments, the catheter base polymer is hydrophobic or partly hydrophobic. A hydrophobic base polymer facilitates increased hydrophobic-hydrophobic interactions between the hydrophobic portion of the additive and the base polymer. This further decreases the energetic favourability for the hydrophobic portion to leave the base polymer and migrate out into the more hydrophilic external environment.

In some embodiments, the base polymer comprises a polymer independently chosen from: polyvinyl chloride, polytetrafluoroethylene, polyolefins, latex, silicones, synthetic rubbers, polyurethanes, polyesters, polyacrylates, polyamides, thermoplastic elastomeric materials, styrene block copolymers, polyether block amide, thermoplastic vulcanizates, thermoplastic copolyesters, thermoplastic polyamides, styrene-butadiene copolymer (SBC), styrene-ethylene-butylene-styrene copolymer (SEBS), and water disintegrable or enzymatically hydrolysable material, or combinations, blends or copolymers of any of the above materials. In some embodiments, the base polymer comprises a polymer independently selected from the group consisting of: polyvinyl chloride, polytetrafluoroethylene, polyolefins, latex, silicones, synthetic rubbers, polyurethanes, polyesters, polyacrylates, polyamides, thermoplastic elastomeric materials, styrene block copolymers, polyether block amide, thermoplastic vulcanizates, thermoplastic copolyesters, thermoplastic polyamides, styrene-butadiene copolymer (SBC), styrene-ethylene-butylene-styrene copolymer (SEBS), and water disintegrable or enzymatically hydrolysable material, or combinations, blends or copolymers of any of the above materials.

In preferred embodiments, the base polymer comprises a polymer independently chosen from: polyolefins, polyesters, polyacrylates, polyamides, thermoplastic elastomeric material, polyether block amide, thermoplastic vulcanizates, thermoplastic copolyesters, thermoplastic polyamides, fluororubber, and water disintegrable or enzymatically hydrolysable material or combinations, blends or copolymers of any of the above materials. In preferred embodiments, the base polymer comprises a polymer independently selected from the group consisting of: polyolefins, polyesters, polyacrylates, polyamides, thermoplastic elastomeric material, polyether block amide, thermoplastic vulcanizates, thermoplastic copolyesters, thermoplastic polyamides, fluororubber, and water disintegrable or enzymatically hydrolysable material or combinations, blends or copolymers of any of the above materials.

In some embodiments, said water disintegrable or enzymatically hydrolysable material comprises a material chosen from: polyvinyl alcohol, extrudable polyvinyl alcohol, polyacrylic acids, polylactic acid, polyesters, polyglycolide, polyglycolic acid, poly lactic-co-glycolic acid, polylactide, amines, polyacrylamides, poly(N-(2-Hydroxypropyl) methacrylamide), starch, modified starches or derivatives, amylopectin, pectin, xanthan, scleroglucan, dextrin, chitosans, chitins, agar, alginate, carrageenans, laminarin, saccharides, polysaccharides, sucrose, polyethylene oxide, polypropylene oxide, acrylics, polyacrylic acid blends, poly(methacrylic acid), polystyrene sulfonate, polyethylene sulfonate, lignin sulfonate, polymethacrylamides, copolymers of aminoalkyl-acrylamides and methacrylamides, melamine-formaldehyde copolymers, vinyl alcohol copolymers, cellulose ethers, poly-ethers, polyethylene oxide, blends of polyethylene-polypropylene glycol, carboxymethyl cellulose, guar gum, locust bean gum, hydroxypropyl cellulose, vinylpyrrolidone polymers and copolymers, polyvinyl pyrrolidone-ethylene-vinyl acetate, polyvinyl pyrrolidone-carboxymethyl cellulose, carboxymethyl cellulose shellac, copolymers of vinylpyrrolidone with vinyl acetate, hydroxyethyl cellulose, gelatin, poly-caprolactone, poly(p-dioxanone), or combinations, blends or co-polymers of any of the above materials. In some embodiments, said water disintegrable or enzymatically hydrolysable material comprises a material of the group consisting of: polyvinyl alcohol, extrudable polyvinyl alcohol, polyacrylic acids, polylactic acid, polyesters, polyglycolide, polyglycolic acid, poly lactic-co-glycolic acid, polylactide, amines, polyacrylamides, poly(N-(2-Hydroxypropyl) methacrylamide), starch, modified starches or derivatives, amylopectin, pectin, xanthan, scleroglucan, dextrin, chitosans, chitins, agar, alginate, carrageenans, laminarin, saccharides, polysaccharides, sucrose, polyethylene oxide, polypropylene oxide, acrylics, polyacrylic acid blends, poly(methacrylic acid), polystyrene sulfonate, polyethylene sulfonate, lignin sulfonate, polymethacrylamides, copolymers of aminoalkyl-acrylamides and methacrylamides, melamine-formaldehyde copolymers, vinyl alcohol copolymers, cellulose ethers, poly-ethers, polyethylene oxide, blends of polyethylene-polypropylene glycol, carboxymethyl cellulose, guar gum, locust bean gum, hydroxypropyl cellulose, vinylpyrrolidone polymers and copolymers, polyvinyl pyrrolidone-ethylene-vinyl acetate, polyvinyl pyrrolidone-carboxymethyl cellulose, carboxymethyl cellulose shellac, copolymers of vinylpyrrolidone with vinyl acetate, hydroxyethyl cellulose, gelatin, poly-caprolactone, poly(p-dioxanone), or combinations, blends or co-polymers of any of the above materials.

In other preferred embodiments, the base polymer comprises a polymer independently chosen from: polyolefins, polyvinyl chloride, polyurethane, styrene-butadiene copolymer (SBC), styrene-ethylene-butylene-styrene copolymer (SEBS), and thermoplastic elastomeric material or combinations, blends or copolymers of any of the above materials. In other preferred embodiments, the base polymer comprises a polymer independently selected from the group consisting of: polyolefins, polyvinyl chloride, polyurethane, styrene-butadiene copolymer (SBC), styrene-ethylene-butylene-styrene copolymer (SEBS), and thermoplastic elastomeric material or combinations, blends or copolymers of any of the above materials.

In some preferred embodiments, the base polymer comprises a polyolefin, especially polyethylene and/or polypropylene.

In some preferred embodiments, the base polymer comprises a thermoplastic elastomeric material. The base polymer may comprise a thermoplastic polyolefin.

The thermoplastic base polymer may comprise a hydrophobic polymer independently chosen from: Accurel™, Styroflex™, Styrolux™, Meliflex™, and Mediprene™ and any combination thereof. The thermoplastic base polymer may comprise a hydrophobic polymer independently selected from the group consisting of: Accurel™, Styroflex™, Styrolux™ MelifleX™, and Mediprene™ and any combination thereof.

The thermoplastic base polymer may comprise Estane™ 58315, which is both hydrophobic and hydrophilic.

The aqueous gel may have a viscosity of at least 1000 cP, or at least 1250, 1500, 1750, 2000, 2250, 2500, 2750, 3000, 3250, 3500, 3750, 4000, 4250, 4500, 4750, or at least 5000 cP. The aqueous gel may have a viscosity of no greater than 5000000, or no greater than 4000000, 3000000, 2000000, 1000000, 900000, or no greater than 800000 cP.

In some embodiments, the aqueous gel has a viscosity of between 1000-1000000 cP, or between 1500-900000, or between 300000-850000, or between 5000-800000, or between 6000-700000, or between 7000-600000, or between 8000-500000, or between 9000-400000 cP. In some embodiments, the gel has a viscosity of between 100000-500000 cP, or between 150000-450000, or between 200000-400000 cP. In some embodiments, the gel has a viscosity of between 8000-32000 cP, or between 9000-31000, or between 10000-30000, or between 11000-29000, or between 12000-28000, or between 13000-27000 cP.

In some preferred embodiments, there is provided a urinary catheter comprising a hollow polymeric tubular body comprising a base polymer and an amphiphilic additive, wherein at least a part of the body is coated with an aqueous gel, and wherein the gel has a viscosity of between 5000-800000 cP.

In some preferred embodiments, there is provided a urinary catheter comprising a hollow polymeric tubular body comprising a base polymer and an amphiphilic additive, wherein at least part of the body is coated with an aqueous gel, wherein the gel comprises water in a total amount of between 5-99 wt. %, and wherein the gel has a viscosity of between 5000-800000 cP.

In some embodiments, the aqueous gel has a pH of at least 1, or at least 2, 3, 4, or at least 5. In some embodiments, the aqueous gel has a pH of no greater than 12, or no greater than 11, 10, 9, or no greater than 8.

In some embodiments, the aqueous gel has a pH of between 3-9, or between 4-8, or between 5-8. In some embodiments, the gel has a pH of between 4-7, or preferably between 5-6. In some embodiments, the gel has a pH of between 5.5-8, or between 6-7.5, or between 6-7.

In some preferred embodiments, there is provided a urinary catheter comprising a hollow polymeric tubular body comprising a base polymer and an amphiphilic additive, wherein at least a part of the body is coated with an aqueous gel, and wherein the gel has a pH of between 4-7.

In some preferred embodiments, there is provided a urinary catheter comprising a hollow polymeric tubular body comprising a base polymer and an amphiphilic additive, wherein at least part of the body is coated with an aqueous gel, wherein the gel comprises water in a total amount of between 5-99 wt. %, and wherein the gel has a pH of between 4-7.

In some embodiments, the aqueous gel comprises water in a total amount of at least 5 wt. %, or in a total amount of at least 10, 15, or in a total amount of at least 20 wt. % of the gel. In some embodiments, the gel comprises water in a total amount of no greater than 99.5 wt. %, 99, 98.5, 98, 97.5, 97, 96.5, 96, 95.5, or in a total amount of no greater than 95 wt. % of the gel.

In some embodiments, the gel comprises water in a total amount of between 5-99 wt. %, or between 10-98 wt. %, or between 20-98 wt. %. In some preferred embodiments, the gel comprises water in a total amount of between 50-98 wt. %, or between 55-98 wt. %, or between 55-95 wt. %, or between 60-95 wt. %, or between 65-95 wt. %, or between 70-95 wt. %, or between 75-95 wt. %.

In some preferred embodiments, the gel comprises water in a total amount of between 20-98 wt. %, or between 50-98 wt. %.

In some preferred embodiments, there is provided a urinary catheter comprising a hollow polymeric tubular body comprising a base polymer and an amphiphilic additive, wherein at least part of the body is coated with an aqueous gel, wherein the gel comprises water in a total amount of between 20-98 wt. %, or between 50-98 wt. %.

In some embodiments, the gel comprises glycerin and/or at least one paraben.

In some preferred embodiments, there is provided a urinary catheter comprising a hollow polymeric tubular body comprising a base polymer and an amphiphilic additive, wherein at least a part of the body is coated with an aqueous gel, and wherein the gel comprises at least one paraben.

In some preferred embodiments, there is provided a urinary catheter comprising a hollow polymeric tubular body comprising a base polymer and an amphiphilic additive, wherein at least part of the body is coated with an aqueous gel, wherein the gel comprises water in a total amount of between 5-99 wt. %, and wherein the gel comprises at least one paraben.

In some embodiments, the gel comprises glycerin in a total amount of at least 1 wt. %, or at least 2, 3, 4, or at least 5 wt. %. In some embodiments, the gel comprises glycerin in a total amount of no greater than 90 wt. %, or no greater than 85, 80, 75, 70, 65, 60, 55, 50, 45, or no greater than 40 wt. %.

In some embodiments, the gel comprises glycerin in a total amount of between 5-80 wt. %, or between 7-77 wt. %, or between 9-75 wt. %. In some preferred embodiments, the gel comprises glycerin in a total amount of between 5-50 wt. %, or between 6-45 wt. %, or between 7-40 wt. %, or between 8-35, 8-30, 8-25, 8-20, or between 8-15 wt. %.

In some embodiments, the gel comprises the paraben in a total amount of at least 0.02 wt. %, or at least 0.04, 0.06, or at least 0.08 wt. %. In some embodiments, the gel comprises the paraben in a total amount of no greater than 1 wt. %, or no greater than 0.8, 0.6, or no greater than 0.4 wt. %.

In some embodiments, the gel comprises the paraben in a total amount of between 0.025-0.5 wt. %, or between 0.05-0.4 wt. %, or between 0.05-0.3 wt. %.

In some preferred embodiments, there is provided a urinary catheter comprising a hollow polymeric tubular body comprising a base polymer and an amphiphilic additive, wherein at least a part of the body is coated with an aqueous gel, and wherein the gel comprises at least one paraben in a total amount of between 0.05-0.3 wt. %.

In some preferred embodiments, there is provided a urinary catheter comprising a hollow polymeric tubular body comprising a base polymer and an amphiphilic additive, wherein at least part of the body is coated with an aqueous gel, wherein the gel comprises water in a total amount of between 5-99 wt. %, and wherein the gel comprises at least one paraben in a total amount of between 0.05-0.3 wt. %.

In some embodiments, at least one paraben comprises at least one alkyl paraben. At least one alkyl paraben may comprise at least one C₁-C₁₀ alkyl paraben, or at least one C₁-C₈alkyl paraben, or preferably at least one C₁-C₅ alkyl paraben. In some embodiments, at least one alkyl paraben is independently chosen from: methylparaben, ethylparaben, propylparaben, butylparaben, and combinations thereof. In some embodiments, at least one alkyl paraben is independently selected from the group consisting of: methylparaben, ethylparaben, propylparaben, butylparaben, and combinations thereof. In preferred embodiments, the gel comprises methylparaben and/or propylparaben.

In some preferred embodiments, there is provided a urinary catheter comprising a hollow polymeric tubular body comprising a base polymer and an amphiphilic additive, wherein at least a part of the body is coated with an aqueous gel, and wherein the gel comprises at least one alkyl paraben, preferably at least one C₁-C₁₀ alkyl paraben.

In some preferred embodiments, there is provided a urinary catheter comprising a hollow polymeric tubular body comprising a base polymer and an amphiphilic additive, wherein at least part of the body is coated with an aqueous gel, wherein the gel comprises water in a total amount of between 5-99 wt. %, and wherein the gel comprises at least one alkyl paraben, preferably at least one C₁-C₁₀ alkyl paraben.

In some preferred embodiments, there is provided a urinary catheter comprising a hollow polymeric tubular body comprising a base polymer and an amphiphilic additive, wherein at least a part of the body is coated with an aqueous gel, and wherein the gel comprises at least one alkyl paraben that is independently selected from the group consisting of: methylparaben, ethylparaben, propylparaben, butylparaben, and combinations thereof.

In some preferred embodiments, there is provided a urinary catheter comprising a hollow polymeric tubular body comprising a base polymer and an amphiphilic additive, wherein at least part of the body is coated with an aqueous gel, wherein the gel comprises water in a total amount of between 5-99 wt. %, and wherein the gel comprises at least one alkyl paraben that is independently selected from the group consisting of: methylparaben, ethylparaben, propylparaben, butylparaben, and combinations thereof.

In some preferred embodiments, there is provided a urinary catheter comprising a hollow polymeric tubular body comprising a base polymer and an amphiphilic additive, wherein at least a part of the body is coated with an aqueous gel, and wherein the gel comprises methylparaben and/or propylparaben.

In some preferred embodiments, there is provided a urinary catheter comprising a hollow polymeric tubular body comprising a base polymer and an amphiphilic additive, wherein at least part of the body is coated with an aqueous gel, wherein the gel comprises water in a total amount of between 5-99 wt. %, and wherein the gel comprises methylparaben and/or propylparaben.

In some embodiments, the gel comprises methylparaben in a total amount of at least 0.01 wt. %, or at least 0.02, 0.03, 0.04, or at least 0.05 wt. %. In some embodiments, the gel comprises methylparaben in a total amount of no greater than 0.3 wt. %, or no greater than 0.29, 0.28, 0.27, 0.26, 0.25, 0.24, 0.23, or no greater than 0.22 wt. %. In some embodiments, the gel comprises methylparaben in a total amount of between 0.03-0.3 wt. %, or between 0.04-0.26 wt. %, or between 0.05-0.25 wt. %.

In some embodiments, the gel comprises propylparaben in a total amount of at least 0.005 wt. %, or at least 0.01, 0.015, or at least 0.02 wt. %. In some embodiments, the gel comprises propylparaben in a total amount of no greater than 0.3 wt. %, or no greater than 0.28, 0.26, 0.24, 0.22, 0.2, or no greater than 0.18, 0.16, 0.14, 0.12, 0.1, 0.08, or no greater than 0.06 wt. %. In some embodiments, the gel comprises propylparaben in a total amount of between 0.005-0.3 wt. %, or between 0.01-0.2, or between 0.01-0.15, or between 0.01-0.1, or between 0.02-0.08 wt. %.

In some embodiments, the gel comprises both glycerin and at least one paraben.

In some preferred embodiments, there is provided a urinary catheter comprising a hollow polymeric tubular body comprising a base polymer and an amphiphilic additive, wherein at least a part of the body is coated with an aqueous gel, and wherein the gel comprises both glycerin and at least one paraben.

In some preferred embodiments, there is provided a urinary catheter comprising a hollow polymeric tubular body comprising a base polymer and an amphiphilic additive, wherein at least part of the body is coated with an aqueous gel, wherein the gel comprises water in a total amount of between 5-99 wt. %, and wherein the gel comprises both glycerin and at least one paraben.

The gel may comprise glycerin and at least one alkyl paraben. The gel may comprise glycerin and at least one C1-C10 alkyl paraben, C1-C8 alkyl paraben, or C1-C5 alkyl paraben. The gel may comprise glycerin and at least one alkyl paraben independently chosen from: methylparaben, ethylparaben, propylparaben, butylparaben, and combinations thereof. The gel may comprise glycerin and at least one alkyl paraben independently selected from the group consisting of: methylparaben, ethylparaben, propylparaben, butylparaben, and combinations thereof. The gel may preferably comprise glycerin and at least one alkyl paraben independently chosen from: methylparaben, propylparaben, and a combination thereof. The gel may preferably comprise glycerin and at least one alkyl paraben independently selected from the group consisting of: methylparaben, propylparaben, and a combination thereof. The gel may comprise glycerin, methylparaben and propylparaben.

In some preferred embodiments, there is provided a urinary catheter comprising a hollow polymeric tubular body comprising a base polymer and an amphiphilic additive, wherein at least a part of the body is coated with an aqueous gel, and wherein the gel comprises glycerin and at least one alkyl paraben independently selected from the group consisting of: methylparaben, propylparaben, and a combination thereof.

In some preferred embodiments, there is provided a urinary catheter comprising a hollow polymeric tubular body comprising a base polymer and an amphiphilic additive, wherein at least part of the body is coated with an aqueous gel, wherein the gel comprises water in a total amount of between 5-99 wt. %, and wherein the gel comprises glycerin and at least one alkyl paraben independently selected from the group consisting of: methylparaben, propylparaben, and a combination thereof.

In some embodiments, the gel comprises the paraben, preferably the at least one alkylparaben in a total amount of between 0.025-0.5 wt. %; and the gel comprises glycerin in a total amount of between 5-80 wt. %, or between 7-77 wt. %, or between 9-75 wt. %. In some embodiments, the gel comprises the paraben, preferably the at least one alkylparaben in a total amount of between 0.025-0.5 wt. %; and the gel comprises glycerin in a total amount of between 5-50 wt. %, or between 6-45 wt. %, or between 7-40 wt. %, or between 8-35, 8-30, 8-25, 8-20, or between 8-15 wt. %.

In some embodiments, the gel comprises the paraben, preferably the at least one alkylparaben in a total amount of between 0.05-0.4 wt. %; and the gel comprises glycerin in a total amount of between 5-80 wt. %, or between 7-77 wt. %, or between 9-75 wt. %. In some embodiments, the gel comprises the paraben, preferably the at least one alkylparaben in a total amount of between 0.05-0.4 wt. %; and the gel comprises glycerin in a total amount of between 5-50 wt. %, or between 6-45 wt. %, or between 7-40 wt. %, or between 8-35, 8-30, 8-25, 8-20, or between 8-15 wt. %.

In some embodiments, the gel comprises the paraben, preferably the at least one alkylparaben in a total amount of between 0.05-0.3 wt. %; and the gel comprises glycerin in a total amount of between 5-80 wt. %, or between 7-77 wt. %, or between 9-75 wt. %. In some embodiments, the gel comprises the paraben, preferably the at least one alkylparaben in a total amount of between 0.05-0.3 wt. %; and the gel comprises glycerin in a total amount of between 5-50 wt. %, or between 6-45 wt. %, or between 7-40 wt. %, or between 8-35, 8-30, 8-25, 8-20, or between 8-15 wt. %.

In some embodiments, the gel comprises methylparaben in a total amount of between 0.03-0.3 wt. %; propylparaben in a total amount of between 0.005-0.3 wt. %; and glycerin in a total amount of between 5-80 wt. %, or between 7-77 wt. %, or between 9-75 wt. %. In some embodiments, the gel comprises methylparaben in a total amount of between 0.03-0.3 wt. %; propylparaben in a total amount of between 0.005-0.3 wt. %; and glycerin in a total amount of between 5-50 wt. %, or between 6-45 wt. %, or between 7-40 wt. %, or between 8-35, 8-30, 8-25, 8-20, or between 8-15 wt. %.

In some embodiments, the gel comprises methylparaben in a total amount of between 0.04-0.26 wt. %; propylparaben in a total amount of between 0.01-0.15 wt. %; and glycerin in a total amount of between 5-80 wt. %, or between 7-77 wt. %, or between 9-75 wt. %. In some embodiments, the gel comprises methylparaben in a total amount of between 0.04-0.26 wt. %; propylparaben in a total amount of between 0.01-0.15 wt. %; and glycerin in a total amount of between 5-50 wt. %, or between 6-45 wt. %, or between 7-40 wt. %, or between 8-35, 8-30, 8-25, 8-20, or between 8-15 wt. %.

In some embodiments, the gel comprises methylparaben in a total amount of between 0.05-0.25 wt. %; propylparaben in a total amount of between 0.02-0.08 wt. %; and glycerin in a total amount of between 5-80 wt. %, or between 7-77 wt. %, or between 9-75 wt. %. In some embodiments, the gel comprises methylparaben in a total amount of between 0.05-0.25 wt. %; propylparaben in a total amount of between 0.02-0.08 wt. %; and glycerin in a total amount of between 5-50 wt. %, or between 6-45 wt. %, or between 7-40 wt. %, or between 8-35, 8-30, 8-25, 8-20, or between 8-15 wt. %.

In some embodiments, the aqueous gel comprises at least one species that is independently chosen from: a chlorine-containing species, a peroxide species, a base, an acid, a photosensitiser, a permanganate species, an alcohol, a phenol, an aldehyde, ionic silver, molecular iodine or an iodophor, an imine-containing species, a salt and combinations thereof.

In some embodiments, the aqueous gel comprises at least one species that is independently selected from the group consisting of: a chlorine-containing species, a peroxide species, a base, an acid, a photosensitiser, a permanganate species, an alcohol, a phenol, an aldehyde, ionic silver, molecular iodine or an iodophor, an imine-containing species, a salt and combinations thereof.

Such species may further allow for effective sterilisation of the catheter, in addition to lubrication.

In some embodiments, the aqueous gel comprises at least one chlorine-containing species independently chosen from: hypochlorous acid, at least one hypochlorite salt, chlorine dioxide, molecular chlorine, a dichloroisocyanurate salt, chloroazodin, dichlorodimethylhydantoin, chloroxylenol, chlorhexidine, and combinations thereof. In some embodiments, the aqueous gel comprises at least one chlorine-containing species independently selected from the group consisting of: hypochlorous acid, at least one hypochlorite salt, chlorine dioxide, molecular chlorine, a dichloroisocyanurate salt, chloroazodin, dichlorodimethylhydantoin, chloroxylenol, chlorhexidine, and combinations thereof.

In some embodiments, the aqueous gel comprises at least one chlorine-containing species independently chosen from: hypochlorous acid, at least one hypochlorite salt, chlorine dioxide, and combinations thereof. In some embodiments, the aqueous gel comprises at least one chlorine-containing species independently selected from the group consisting of: hypochlorous acid, at least one hypochlorite salt, chlorine dioxide, and combinations thereof.

In some embodiments, the gel comprises the chlorine-containing species in a total concentration of at least 0.005 wt. % of the gel, or at least 0.006, 0.007, 0.008, 0.009, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.11, 0.12, 0.13, 0.14, 0.15, 0.16, 0.17, 0.18, 0.19, or at least 0.2 wt. % of the gel.

In some embodiments, the gel comprises the chlorine-containing species in a total concentration of no greater than 5 wt. % of the gel, or no greater than 4.5, 4, 3.5, 3, 2.5, 2, 1.5, 1, 0.9, 0.8, 0.7, 0.6, 0.5, 0.4, 0.3, or no greater than 0.2 wt. % of the gel.

In some embodiments, the gel comprises the chlorine-containing species in a total concentration of between 0.005-1.5 wt. % of the gel, or between 0.005-1, or between 0.005-0.6 wt. % of the gel, or between 0.01-0.5, 0.015-0.4, 0.02-0.3, or between 0.025-0.2 wt. % of the gel, or preferably between 0.025-0.1 wt. % of the gel.

In some embodiments, the total concentration of chlorine-containing species in the gel is between 0.05-0.6 wt. % of the gel, or between 0.01-0.5, 0.015-0.4, 0.02-0.3, or between 0.025-0.2 wt. % of the gel, or between 0.025-0.1 wt. %.

In some embodiments, the gel comprises glycerin, at least one chlorine-containing species, and at least one paraben. The gel may comprise glycerin, at least one chlorine-containing species, and at least one alkyl paraben. The gel may comprise glycerin, at least one chlorine-containing species, and at least one C1-C10 alkyl paraben, C1-C8 alkyl paraben, or C1-C5 alkyl paraben. The gel may comprise glycerin, at least one chlorine-containing species, and at least one alkyl paraben independently chosen from: methylparaben, ethylparaben, propylparaben, butylparaben, and combinations thereof. The gel may comprise glycerin, at least one chlorine-containing species, and at least one alkyl paraben independently selected from the group consisting of: methylparaben, ethylparaben, propylparaben, butylparaben, and combinations thereof. The gel may comprise glycerin, at least one chlorine-containing species, and at least one alkyl paraben independently chosen from: methylparaben, propylparaben, and a combination thereof. The gel may comprise glycerin, at least one chlorine-containing species, and at least one alkyl paraben independently selected from the group consisting of: methylparaben, propylparaben, and a combination thereof. The gel may comprise glycerin, at least one chlorine-containing species, methylparaben and propylparaben. In such embodiments, at least one chlorine-containing species may be independently chosen from: hypochlorous acid, at least one hypochlorite salt, chlorine dioxide, molecular chlorine, a dichloroisocyanurate salt, chloroazodin, dichlorodimethylhydantoin, chloroxylenol, chlorhexidine, and combinations thereof. In such embodiments, at least one chlorine-containing species may be independently selected from the group consisting of: hypochlorous acid, at least one hypochlorite salt, chlorine dioxide, molecular chlorine, a dichloroisocyanurate salt, chloroazodin, dichlorodimethylhydantoin, chloroxylenol, chlorhexidine, and combinations thereof. At least one chlorine-containing species may be independently chosen from: hypochlorous acid, at least one hypochlorite salt, chlorine dioxide, and combinations thereof. At least one chlorine-containing species may be independently selected from the group consisting of: hypochlorous acid, at least one hypochlorite salt, chlorine dioxide, and combinations thereof.

In some embodiments, the gel comprises the paraben, preferably the at least one alkyl paraben in a total amount of between 0.025-0.5 wt. %; the chlorine-containing species in a total amount of between 0.005-1.5 wt. %; and glycerin in a total amount of between 5-80 wt. %, or between 7-77 wt. %, or between 9-75 wt. %. In some embodiments, the gel comprises the paraben, preferably the at least one alkyl paraben in a total amount of between 0.025-0.5 wt. %; the chlorine-containing species in a total amount of between 0.005-1.5 wt. %; and glycerin in a total amount of between 5-50 wt. %, or between 6-45 wt. %, or between 7-40 wt. %, or between 8-35, 8-30, 8-25, 8-20, or between 8-15 wt. %.

In some embodiments, the gel comprises the paraben, preferably the at least one alkyl paraben in a total amount of between 0.05-0.4 wt. %; the chlorine-containing species in a total amount of between 0.025-0.2 wt. %; and glycerin in a total amount of between 5-80 wt. %, or between 7-77 wt. %, or between 9-75 wt. %. In some embodiments, the gel comprises the paraben, preferably the at least one alkyl paraben in a total amount of between 0.05-0.4 wt. %; the chlorine-containing species in a total amount of between 0.025-0.2 wt. %; and glycerin in a total amount of between 5-50 wt. %, or between 6-45 wt. %, or between 7-40 wt. %, or between 8-35, 8-30, 8-25, 8-20, or between 8-15 wt. %.

In some embodiments, the gel comprises the paraben, preferably the at least one alkyl paraben in a total amount of between 0.05-0.3 wt. %; the chlorine-containing species in a total amount of between 0.025-0.1 wt. %; and glycerin in a total amount of between 5-80 wt. %, or between 7-77 wt. %, or between 9-75 wt. %. In some embodiments, the gel comprises the paraben, preferably the at least one alkyl paraben in a total amount of between 0.05-0.3 wt. %; the chlorine-containing species in a total amount of between 0.025-0.1 wt. %; and glycerin in a total amount of between 5-50 wt. %, or between 6-45 wt. %, or between 7-40 wt. %, or between 8-35, 8-30, 8-25, 8-20, or between 8-15 wt. %.

In some embodiments, the gel comprises at least one hypochlorite salt. At least one hypochlorite salt may preferably comprise a countercation. The countercation may comprise an inorganic countercation. The countercation may comprise a metal countercation. In some embodiments, at least one countercation is independently chosen from: an alkali metal cation, an alkaline earth metal cation, a group III metal cation, a transition metal cation, an ammonium cation, an aromatic nitrogen-based cation, and combinations thereof. In some embodiments, at least one countercation is independently selected from the group consisting of: an alkali metal cation, an alkaline earth metal cation, a group III metal cation, a transition metal cation, an ammonium cation, an aromatic nitrogen-based cation, and combinations thereof. At least one countercation may be independently chosen from: ammonium, calcium, iron, magnesium, potassium, pyridinium, quaternary ammonium, sodium, copper, aluminium, lithium, beryllium, strontium, and zinc. At least one countercation may be independently selected from the group consisting of: ammonium, calcium, iron, magnesium, potassium, pyridinium, quaternary ammonium, sodium, copper, aluminium, lithium, beryllium, strontium, and zinc. At least one countercation may preferably be an alkali metal cation or an alkaline earth metal cation. At least one countercation may preferably be independently chosen from: calcium, lithium, and sodium. At least one countercation may preferably be independently selected from the group consisting of: calcium, lithium, and sodium. In preferred embodiments, at least one hypochlorite salt is sodium hypochlorite.

In some embodiments, the gel comprises hypochlorous acid and at least one hypochlorite salt. At least one hypochlorite salt may preferably be as described in statements of invention above. The gel may preferably comprise hypochlorous acid and at least one alkali metal hypochlorite salt. In some embodiments, the gel comprises hypochlorous acid and sodium hypochlorite.

In some embodiments, the gel comprises chlorine dioxide and at least one of: hypochlorous acid and at least one hypochlorite salt. In some embodiments, the gel comprises chlorine dioxide, hypochlorous acid, and at least one hypochlorite salt. In such embodiments, at least one hypochlorite salt may preferably be as described in statements of invention above. At least one hypochlorite salt may preferably be an alkali metal hypochlorite salt, which may comprise sodium hypochlorite.

In some embodiments, the gel comprises chlorine dioxide in a total concentration of at least 0.005 wt. % of the gel, or at least 0.006, 0.007, 0.008, 0.009, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.11, 0.12, 0.13, 0.14, 0.15, 0.16, 0.17, 0.18, 0.19, or at least 0.2 wt. % of the gel.

In some embodiments, the gel comprises chlorine dioxide in a total combined concentration of no greater than 5 wt. % of the gel, or no greater than 4.5, 4, 3.5, 3, 2.5, 2, 1.5, 1, 0.9, 0.8, 0.7, 0.6, 0.5, 0.4, 0.3, or no greater than 0.2 wt. % of the gel.

In some embodiments, the gel comprises chlorine dioxide in a total combined concentration of between 0.005-1.5 wt. % of the gel, or between 0.005-1, or between 0.005-0.6 wt. % of the gel, or between 0.01-0.5, 0.015-0.4, 0.02-0.3, or between 0.025-0.2 wt. % of the gel, or preferably between 0.025-0.1 wt. % of the gel.

In some embodiments, the gel comprises one or more of: chlorine dioxide, hypochlorous acid, and at least one hypochlorite in a total combined concentration of at least 0.005 wt. % of the gel, or at least 0.006, 0.007, 0.008, 0.009, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.11, 0.12, 0.13, 0.14, 0.15, 0.16, 0.17, 0.18, 0.19, or at least 0.2 wt. % of the gel.

In some embodiments, the gel comprises one or more of: chlorine dioxide, hypochlorous acid, and at least one hypochlorite salt in a total combined concentration of no greater than 5 wt. % of the gel, or no greater than 4.5, 4, 3.5, 3, 2.5, 2, 1.5, 1, 0.9, 0.8, 0.7, 0.6, 0.5, 0.4, 0.3, or no greater than 0.2 wt. % of the gel.

In some embodiments, the gel comprises one or more of: chlorine dioxide, hypochlorous acid, and at least one hypochlorite salt in a total combined concentration of between 0.005-1.5 wt. % of the gel, or between 0.005-1, or between 0.005-0.6 wt. % of the gel, or between 0.01-0.5, 0.015-0.4, 0.02-0.3, or between 0.025-0.2 wt. % of the gel, or preferably between 0.025-0.1 wt. % of the gel.

In some embodiments, the gel comprises hypochlorous acid and/or at least one hypochlorite in a total combined concentration of at least 0.005 wt. % of the gel, or at least 0.006, 0.007, 0.008, 0.009, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.11, 0.12, 0.13, 0.14, 0.15, 0.16, 0.17, 0.18, 0.19, or at least 0.2 wt. % of the gel.

In some embodiments, the gel comprises hypochlorous acid and/or at least one hypochlorite salt in a total combined concentration of no greater than 5 wt. % of the gel, or no greater than 4.5, 4, 3.5, 3, 2.5, 2, 1.5, 1, 0.9, 0.8, 0.7, 0.6, 0.5, 0.4, 0.3, or no greater than 0.2 wt. % of the gel.

In some embodiments, the gel comprises hypochlorous acid and/or at least one hypochlorite salt in a total combined concentration of between 0.005-1.5 wt. % of the gel, or between 0.005-1, or between 0.005-0.6 wt. % of the gel, or between 0.01-0.5, 0.015-0.4, 0.02-0.3, or between 0.025-0.2 wt. % of the gel, or preferably between 0.025-0.1 wt. % of the gel.

In some embodiments, the gel comprises hypochlorous acid in a total concentration of at least 0.005 wt. % of the gel, or at least 0.006, 0.007, 0.008, 0.009, 0.01, 0.015, 0.02, 0.025, 0.03, 0.035, 0.04, 0.045, 0.05, 0.055, 0.06, or preferably at least 0.065, 0.07, 0.075, 0.08, 0.085, 0.09, 0.095, 0.1, 0.11, 0.12, 0.13, 0.14, 0.15, 0.16, 0.17, 0.18, 0.19, or at least 0.2 wt. % of the gel.

In some embodiments, the gel comprises hypochlorous acid in a total concentration of no greater than 5 wt. % of the gel, or no greater than 4.5, 4, 3.5, 3, 2.5, 2, 1.5, 1, 0.9, 0.8, 0.7, 0.6, 0.5, 0.4, 0.3, or no greater than 0.2 wt. % of the gel.

In some embodiments, the gel comprises hypochlorous acid in a total concentration of between 0.005-1.5 wt. % of the gel, or between 0.005-1, or between 0.005-0.6 wt. % of the gel, or between 0.01-0.5, 0.015-0.4, 0.02-0.3, or between 0.025-0.2 wt. % of the gel, or between 0.025-0.1 wt. % of the gel.

The gel may comprise the hypochlorite salt in a total concentration of at least 0.005 wt. % of the gel, or at least 0.006, 0.007, 0.008, 0.009, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.11, 0.12, 0.13, 0.14, 0.15, 0.16, 0.17, 0.18, 0.19, or at least 0.2 wt. % of the gel.

In some embodiments, the gel comprises the hypochlorite salt in a total concentration of no greater than 5 wt. % of the gel, or no greater than 4.5, 4, 3.5, 3, 2.5, 2, 1.5, 1, 0.9, 0.8, 0.7, 0.6, 0.5, 0.4, 0.3, or no greater than 0.2 wt. % of the gel.

In some embodiments, the gel comprises the hypochlorite salt in a total concentration of between 0.005-1.5 wt. % of the gel, or between 0.005-1, or between 0.005-0.6 wt. % of the gel, or between 0.01-0.5, 0.015-0.4, 0.02-0.3, or between 0.025-0.2 wt. % of the gel, or between 0.025-0.1 wt. % of the gel.

In some embodiments, the gel comprises molecular chlorine. In some embodiments, the gel comprises at least one hypochlorite salt and molecular chlorine. In preferred embodiments, the gel comprises hypochlorous acid and molecular chlorine. In some embodiments, the gel comprises hypochlorous acid, molecular chlorine, and at least one hypochlorite salt.

The gel may comprise at least one further salt. At least one salt may comprise a cation that is independently chosen from: ammonium, calcium, iron, magnesium, potassium, pyridinium, quaternary ammonium, sodium, copper, aluminium, lithium, beryllium, strontium, and zinc. At least one salt may comprise a cation that is independently selected from the group consisting of: ammonium, calcium, iron, magnesium, potassium, pyridinium, quaternary ammonium, sodium, copper, aluminium, lithium, beryllium, strontium, and zinc. At least one salt may preferably comprise an alkali metal cation. At least one salt may comprise an anion that is independently chosen from: acetate, carbonate, bicarbonate, chloride, citrate, glutamate, fluoride, bromide, iodide, nitrate, nitrite, oxide, phosphate, ferrocyanide, silicate, gluconate, and sulfate. At least one salt may comprise an anion that is independently selected from the group consisting of: acetate, carbonate, bicarbonate, chloride, citrate, glutamate, fluoride, bromide, iodide, nitrate, nitrite, oxide, phosphate, ferrocyanide, silicate, gluconate, and sulfate. At least one salt may preferably comprise a halide anion.

At least one of the further salts may be independently chosen from: sodium chloride, potassium chloride, calcium chloride, magnesium chloride, calcium chloride, sodium nitrite, magnesium nitrate, calcium nitrate, potassium carbonate, sodium carbonate, sodium bicarbonate, potassium iodide, copper iodide, sodium ferrocyanide, monosodium glutamate, calcium silicate, sodium citrate, potassium citrate, sodium phosphate, potassium phosphate, sodium sulfate, calcium sulfate, sodium gluconate, calcium gluconate, potassium gluconate, sodium acetate, and potassium acetate. At least one of the further salts may be independently selected from the group consisting of: sodium chloride, potassium chloride, calcium chloride, magnesium chloride, calcium chloride, sodium nitrite, magnesium nitrate, calcium nitrate, potassium carbonate, sodium carbonate, sodium bicarbonate, potassium iodide, copper iodide, sodium ferrocyanide, monosodium glutamate, calcium silicate, sodium citrate, potassium citrate, sodium phosphate, potassium phosphate, sodium sulfate, calcium sulfate, sodium gluconate, calcium gluconate, potassium gluconate, sodium acetate, and potassium acetate.

At least one of the further salts may preferably be an alkali metal halide. At least one salt may be independently chosen from: an alkali metal chloride, an alkali metal bromide, an alkali metal iodide, and combinations thereof. At least one salt may be independently selected from the group consisting of: an alkali metal chloride, an alkali metal bromide, an alkali metal iodide, and combinations thereof.

Preferably, the gel may comprise an alkali metal salt and a hypochlorite salt. The gel may comprise a halide salt and a hypochlorite salt. The gel may comprise an alkali metal halide and a hypochlorite salt.

In preferred embodiments, the gel comprises sodium chloride. The gel may preferably comprise a hypochlorite salt and sodium chloride, preferably an alkali metal hypochlorite and sodium chloride, and more preferably sodium hypochlorite and sodium chloride.

In some embodiments, the total concentration of further salt in the gel is between 5-30 wt. %, or of between 10-25, or of between 10-20 wt. % of the gel.

In some embodiments, the gel comprises the hypochlorite salt in a total concentration of between 0.25-2 wt. %, or 0.5-1.5 wt. % of the gel, and the further salt in a total concentration of between 5-30, 10-25, or between 10-20 wt. % of the gel.

In some embodiments, the ratio of the total concentration of further salt to hypochlorite salt in the gel is at least 5, or at least 10, or at least 15. The ratio of the total concentration of further salt to hypochlorite salt in the gel may be between 5-30, or between 10-25, or between 10-20.

In some embodiments, the gel further comprises at least one base.

At least one base may comprise an inorganic base. At least one inorganic base may be independently chosen from: a hydroxide base, a carbonate base, a bicarbonate base, and combinations thereof. At least one inorganic base may be independently selected from the group consisting of: a hydroxide base, a carbonate base, a bicarbonate base, and combinations thereof. In some preferred embodiments, at least one base is a hydroxide base. At least one hydroxide base may be independently chosen from: an alkali metal hydroxide, an alkaline earth metal hydroxide, a group III metal hydroxide, a transition metal hydroxide, and combinations thereof. At least one hydroxide base may be independently selected from the group consisting of: an alkali metal hydroxide, an alkaline earth metal hydroxide, a group III metal hydroxide, a transition metal hydroxide, and combinations thereof. At least one hydroxide base may preferably comprise an alkali metal hydroxide and/or an alkaline earth metal hydroxide. In some embodiments, at least one base comprises an alkali metal hydroxide that is independently chosen from: lithium hydroxide, sodium hydroxide, potassium hydroxide, and combinations thereof. In some embodiments, at least one base comprises an alkali metal hydroxide that is independently selected from the group consisting of: lithium hydroxide, sodium hydroxide, potassium hydroxide, and combinations thereof.

Preferably, the gel may comprise an alkali metal base and a hypochlorite salt. The gel may comprise a hydroxide base and a hypochlorite salt. The gel may comprise an alkali metal hydroxide and a hypochlorite salt.

In a particularly preferred embodiment, the base comprises sodium hydroxide. The gel may preferably comprise a hypochlorite salt and sodium hydroxide, preferably an alkali metal hypochlorite and sodium hydroxide, and more preferably sodium hypochlorite and sodium hydroxide.

In some embodiments, the gel comprises at least one acid that is a carboxylic acid. In some embodiments, the gel comprises at least one acid that is independently chosen from: acetic acid, citric acid, peracetic acid, diperoxy dodecanoic acid, and combinations thereof. In some embodiments, the gel comprises at least one acid that is independently selected from the group consisting of: acetic acid, citric acid, peracetic acid, diperoxy dodecanoic acid, and combinations thereof.

In some embodiments, the gel comprises at least one C1-C10 alcohol, or at least one C1-C5 alcohol, or at least one C1-C3 alcohol. In some embodiments, the gel comprises at least one alcohol that is independently chosen from: ethanol, isopropanol, and combinations thereof. In some embodiments, the gel comprises at least one alcohol that is independently selected from the group consisting of: ethanol, isopropanol, and combinations thereof.

In some embodiments, the gel comprises at least one phenol that is independently chosen from: phenol, thymol, chloroxylenol, and combinations thereof. In some embodiments, the gel comprises at least one phenol that is independently selected from the group consisting of: phenol, thymol, chloroxylenol, and combinations thereof.

In some embodiments, the gel comprises at least one aldehyde that is independently chosen from: glutaraldehyde, noxytiolin, and combinations thereof. In some embodiments, the gel comprises at least one aldehyde that is independently selected from the group consisting of: glutaraldehyde, noxytiolin, and combinations thereof.

In some embodiments, the gel comprises at least one imine-containing species that is independently selected from: polyhexanide, octenidine, and combinations thereof.

The gel may comprise at least one poly (alkylene glycol). At least one poly (alkylene glycol) may be independently chosen from: polyethylene glycol, polypropylene glycol, polybutylene glycol, and combinations thereof. At least one poly (alkylene glycol) may be independently selected from the group consisting of: polyethylene glycol, polypropylene glycol, polybutylene glycol, and combinations thereof. The gel may preferably comprise polyethylene glycol.

In some embodiments, the gel may comprise at least one hydroxyalkylcellulose. In some embodiments, the gel comprises at least one hydroxyalkylcellulose that is independently chosen from: hydroxymethylcellulose, hydroxyethylcellulose, hydroxypropylcellulose, hydroxypropylmethylcellulose, hydroxybutylcellulose, hydroxyethylmethylcellulose, hydroxypropylethylcellulose, hydroxybutylethylcellulose, and combinations thereof. In some embodiments, the gel comprises at least one hydroxyalkylcellulose that is independently selected from the group consisting of: hydroxymethylcellulose, hydroxyethylcellulose, hydroxypropylcellulose, hydroxypropylmethylcellulose, hydroxybutylcellulose, hydroxyethylmethylcellulose, hydroxypropylethylcellulose, hydroxybutylethylcellulose, and combinations thereof. In preferred embodiments, the gel comprises hydroxyethylcellulose.

In some preferred embodiments, there is provided a urinary catheter comprising a hollow polymeric tubular body comprising a base polymer and an amphiphilic additive, wherein at least a part of the body is coated with an aqueous gel, and wherein the gel comprises at least one hydroxyalkylcellulose, preferably comprising hydroxyethylcellulose.

In some preferred embodiments, there is provided a urinary catheter comprising a hollow polymeric tubular body comprising a base polymer and an amphiphilic additive, wherein at least part of the body is coated with an aqueous gel, wherein the gel comprises water in a total amount of between 5-99 wt. %, and wherein the gel comprises at least one hydroxyalkylcellulose, preferably comprising hydroxyethylcellulose.

The gel may comprise the hydroxyalkylcellulose, preferably hydroxyethylcellulose, in a total amount of between 0.25-5 wt. %, or between 0.5-4 wt. %, or between 1-3 wt. %.

In some embodiments, the gel comprises at least one polybiguanide. At least one polybiguanide may be independently chosen from: polyhexamethylene biguanide, polyaminopropyl biguanide, polyhexanide, polyquaternium-10, polyhexamethylene guanidine, and combinations thereof. At least one polybiguanide may be independently selected from the group consisting of: polyhexamethylene biguanide, polyaminopropyl biguanide, polyhexanide, polyquaternium-10, polyhexamethylene guanidine, and combinations thereof. In preferred embodiments, the gel comprises polyhexamethylene biguanide.

The gel may comprise the polybiguanide, preferably polyhexamethylene biguanide, in a total amount of between 0.05-0.5 wt. %, or between 0.075-0.4, or between 0.1-0.3, or between 0.1-0.25 wt. %.

In some embodiments, the gel comprises at least one local-anaesthetic compound, preferably an amide-based local anaesthetic. At least one amide-based local anaesthetic may preferably comprise lidocaine.

The gel may comprise the amide-based local anaesthetic, preferably lidocaine, in a total amount of between 0.5-5 wt. %, or between 1-4 wt. %, or between 1-3 wt. %.

In some embodiments, the aqueous gel is present in a total amount of at least 0.05 wt. % of the combination of the catheter and gel, or of at least 0.1, 0.15, 0.2, 0.25, 0.35, 0.4, 0.45, 0.5, 0.55, 0.6, 0.65, 0.7, 0.75, 0.8, 0.85, 0.9, 0.95, 1, 2, 3, 4, 5, 6, 7, 8, 9, 10, 15, or at least 20 wt. % of the combination of the catheter and gel.

The gel may be present in a total amount of no greater than 40 wt. % of the combination of the catheter and gel, or no greater than 35, 30, 25, 20, 15, 10, or no greater than 5 wt. % of the combination of the catheter and gel.

The gel may be present in a total amount of between 0.1-20 wt. % of the combination of the catheter and gel, or between 0.5-15, or between 0.5-5 wt. % of the combination of the catheter and gel.

In preferred embodiments, at least part of a surface of the catheter body is coated with the aqueous gel. At least part of an outer surface of the body, an inner surface of the body, or both are coated with the gel. The inner surface of the catheter body may comprise a lumen of the catheter. In preferred embodiments, at least part of the outer surface of the catheter body is coated with the gel. The outer surface may preferably be as described in statements of invention above.

In some embodiments, at least 50% of the or each surface area of the catheter body is coated with the aqueous gel, or at least 55, 60, 65, 70, 75, 80, 85, 90, 95, 96, 97, 98, or at least 99% of the or each surface area of the catheter body, preferably at least 75%, or at least 90% of the or each surface area, or between 75% and 100% of the or each surface area is coated with the aqueous gel. In embodiments in which the gel coats both an inner and outer surface of the catheter body, the gel may coat at least 50, 55, 60, 65, 70, 75, 80, 85, 90, 95, 96, 97, 98 or at least 99% of each surface area of the catheter body, preferably at least 75% or at least 90% of each surface area or between 75% and 100% of each surface area of both surfaces.

In some embodiments, the aqueous gel is present as a layer on at least part of the catheter body. The aqueous gel is preferably present as a layer on at least part of the outer surface of the catheter body.

In some embodiments, the layer of gel has a thickness of at least 1 μm, or of at least 2, 3, 4, 5, 6, 7, 8, 9, 10, 15, 20, 25, 30, 35, 40, 45, or of at least 50 μm.

In some embodiments, the layer of gel has a thickness of no greater than 10000 μm, or of no greater than 9000, 8000, 7000, 6000, 5000, 4000, 3000, 2000, 1000, 900, 800, 700, 600, 500, 400, or of no greater than 300 μm.

In some embodiments, the layer of gel has a thickness of between 50-300 μm.

The layer of gel may have a thickness of between 60-300 μm, or of between 80-300, 100-300, 120-300, 140-300, 160-300, 180-300, 200-300, 220-300, 240-300, 260-300, or of between 280-300 μm.

The layer of gel may have a thickness of between 50-280 μm, or of between 50-260, 50-240, 50-220, 50-200, 50-180, 50-160, 50-140, 50-120, 50-100, 50-80, or of between 50-60 μm.

The layer of gel may have a thickness of between 60-280 μm, or of between 80-280, 100-280, 120-280, 140-280, 160-280, 180-280, 200-280, 220-280, 240-280, 260-280, 60-260, 80-260, 100-260, 120-260, 140-260, 160-260, 180-260, 200-260, 220-260, 240-260, 60-240, 80-240, 100-240, 120-240, 140-240, 160-240, 180-240, 200-240, 220-240, 60-220, 80-220, 100-220, 120-220, 140-220, 160-220, 180-220, 200-220, 60-200, 80-200, 100-200, 120-200, 140-200, 160-200, 180-200, 60-180, 80-180, 100-180, 120-180, 140-180, 160-180, 60-160, 80-160, 100-160, 120-160, 140-160, 60-140, 80-140, 100-140, 120-140, 60-120, 80-120, 100-120, 60-100, 80-100, or of between 60-80 μm.

In some embodiments, the layer of gel has a constant thickness that is the same across the entire surface area of the layer. In other embodiments, the layer of gel is thicker in certain regions of the layer than in others. In some embodiments, the layer of gel has a greater thickness towards an end of the catheter body that is to be inserted first into the body, in use, compared to an end of the body that is to be inserted afterwards.

According to a third aspect of the invention, there is provided a urinary catheter kit comprising a container comprising: a urinary catheter comprising a hollow polymeric tubular body comprising a base polymer and an amphiphilic additive; and an aqueous gel lubricating and/or wetting agent.

According to a fourth aspect of the invention, there is provided a urinary catheter kit comprising a container comprising: a urinary catheter comprising a hollow polymeric tubular body comprising a base polymer and an amphiphilic additive; and an aqueous gel lubricating and/or wetting agent, wherein the aqueous gel comprises water in a total amount of between 5-99 wt. %.

The catheter of the third and fourth aspects of the invention is preferably the catheter of the first aspect of the invention. The aqueous gel of the third aspect of the invention is preferably the aqueous gel of the first aspect of the invention. The aqueous gel of the fourth aspect of the invention is preferably the aqueous gel of the second aspect of the invention.

Statements of invention above relating to the first and second aspects of the invention may also be applied mutatis mutandis to the third and fourth aspects of the invention. Statements of invention relating to any other aspect of the invention may also be applied mutatis mutandis to the third and fourth aspects of the invention.

The following statements apply to the third and fourth aspects of the invention.

In some embodiments, the gel is in direct contact with the catheter. The gel may be in direct contact with at least one surface of the catheter body. The at least one surface may comprise an outer surface of the body. The gel may cover at least part of the outer surface of the catheter body.

The at least one surface may comprise an inner surface of the catheter body, and the gel may cover at least part of the inner surface.

At least part of the catheter body may be coated with the aqueous gel, preferably as described for the first aspect of the invention.

In some embodiments, the catheter is submerged in the gel. The catheter may be fully submerged in the gel. In such embodiments, the gel may comprise at least 30% of the internal volume of the container, or at least 40, 50, 60, 70, 80, 90, or at least 95% of the internal volume of the container.

In some embodiments, the catheter is not fully submerged in the gel. In such embodiments, the gel may comprise no greater than 30% of the internal volume of the container, or no greater than 25, 20, 15, 10, 5, 4, 3, 2, or no greater than 1% of the internal volume of the container. In such embodiments, the gel may be in direct contact with at least part of the catheter, preferably with at least one surface thereof. In some embodiments, the gel may be able to move freely within the container.

In some embodiments, the gel may not be in direct contact with the catheter. The gel may be contained in a separate gel container that is stored in the catheter container. The gel container may be a bag or sachet. In some embodiments, the gel container is pierceable, in use, to release the contained gel from the gel container and into direct contact with the catheter, preferably without requiring opening of the catheter container.

In some embodiments, opening the catheter container, in use, brings the gel into direct contact with the catheter. The gel container may be configured to rupture or break to release the gel upon opening of the catheter container. In some embodiments, opening the catheter container, in use, pierces the gel container and releases the contained gel from the gel container and into direct contact with the catheter.

In some embodiments, prior to opening the catheter container, prior to removing the catheter and/or prior to inserting the catheter, the user may release the gel from the gel container and apply the gel to the outer surface of the catheter.

In some preferred embodiments, there is provided a urinary catheter kit comprising a container comprising: a urinary catheter comprising a hollow polymeric tubular body comprising a base polymer and an amphiphilic additive; and an aqueous gel lubricating and/or wetting agent, wherein the gel has a viscosity of between 5000-800000 cP.

In some preferred embodiments, there is provided a urinary catheter kit comprising a container comprising: a urinary catheter comprising a hollow polymeric tubular body comprising a base polymer and an amphiphilic additive; and an aqueous gel lubricating and/or wetting agent, wherein the aqueous gel comprises water in a total amount of between 5-99 wt. %, and wherein the gel has a viscosity of between 5000-800000 cP.

In some preferred embodiments, there is provided a urinary catheter kit comprising a container comprising: a urinary catheter comprising a hollow polymeric tubular body comprising a base polymer and an amphiphilic additive; and an aqueous gel lubricating and/or wetting agent, wherein the gel has a pH of between 4-7.

In some preferred embodiments, there is provided a urinary catheter kit comprising a container comprising: a urinary catheter comprising a hollow polymeric tubular body comprising a base polymer and an amphiphilic additive; and an aqueous gel lubricating and/or wetting agent, wherein the aqueous gel comprises water in a total amount of between 5-99 wt. %, and wherein the gel has a pH of between 4-7.

In some preferred embodiments, there is provided a urinary catheter kit comprising a container comprising: a urinary catheter comprising a hollow polymeric tubular body comprising a base polymer and an amphiphilic additive; and an aqueous gel lubricating and/or wetting agent, wherein the gel comprises at least one paraben.

In some preferred embodiments, there is provided a urinary catheter kit comprising a container comprising: a urinary catheter comprising a hollow polymeric tubular body comprising a base polymer and an amphiphilic additive; and an aqueous gel lubricating and/or wetting agent, wherein the aqueous gel comprises water in a total amount of between 5-99 wt. %, and wherein the gel comprises at least one paraben.

In some preferred embodiments, there is provided a urinary catheter kit comprising a container comprising: a urinary catheter comprising a hollow polymeric tubular body comprising a base polymer and an amphiphilic additive; and an aqueous gel lubricating and/or wetting agent, wherein the gel comprises glycerin and at least one paraben.

In some preferred embodiments, there is provided a urinary catheter kit comprising a container comprising: a urinary catheter comprising a hollow polymeric tubular body comprising a base polymer and an amphiphilic additive; and an aqueous gel lubricating and/or wetting agent, wherein the aqueous gel comprises water in a total amount of between 5-99 wt. %, and wherein the gel comprises glycerin and at least one paraben.

In some preferred embodiments, there is provided a urinary catheter kit comprising a container comprising: a urinary catheter comprising a hollow polymeric tubular body comprising a base polymer and an amphiphilic additive; and an aqueous gel lubricating and/or wetting agent, wherein the gel comprises at least one paraben in a total amount of between 0.05-0.3 wt. %.

In some preferred embodiments, there is provided a urinary catheter kit comprising a container comprising: a urinary catheter comprising a hollow polymeric tubular body comprising a base polymer and an amphiphilic additive; and an aqueous gel lubricating and/or wetting agent, wherein the aqueous gel comprises water in a total amount of between 5-99 wt. %, and wherein the gel comprises at least one paraben in a total amount of between 0.05-0.3 wt. %.

In some preferred embodiments, there is provided a urinary catheter kit comprising a container comprising: a urinary catheter comprising a hollow polymeric tubular body comprising a base polymer and an amphiphilic additive; and an aqueous gel lubricating and/or wetting agent, wherein the gel comprises at least one paraben comprising at least one alkyl paraben, preferably at least one C₁-C₁₀ alkyl paraben.

In some preferred embodiments, there is provided a urinary catheter kit comprising a container comprising: a urinary catheter comprising a hollow polymeric tubular body comprising a base polymer and an amphiphilic additive; and an aqueous gel lubricating and/or wetting agent, wherein the aqueous gel comprises water in a total amount of between 5-99 wt. %, and wherein the gel comprises at least one paraben comprising at least one alkyl paraben, preferably at least one C₁-C₁₀ alkyl paraben.

In some preferred embodiments, there is provided a urinary catheter kit comprising a container comprising: a urinary catheter comprising a hollow polymeric tubular body comprising a base polymer and an amphiphilic additive; and an aqueous gel lubricating and/or wetting agent, wherein the gel comprises at least one paraben comprising at least one alkyl paraben that is independently selected from the group consisting of: methylparaben, ethylparaben, propylparaben, butylparaben, and combinations thereof.

In some preferred embodiments, there is provided a urinary catheter kit comprising a container comprising: a urinary catheter comprising a hollow polymeric tubular body comprising a base polymer and an amphiphilic additive; and an aqueous gel lubricating and/or wetting agent, wherein the aqueous gel comprises water in a total amount of between 5-99 wt. %, and wherein the gel comprises at least one paraben comprising at least one alkyl paraben that is independently selected from the group consisting of: methylparaben, ethylparaben, propylparaben, butylparaben, and combinations thereof.

In some preferred embodiments, there is provided a urinary catheter kit comprising a container comprising: a urinary catheter comprising a hollow polymeric tubular body comprising a base polymer and an amphiphilic additive; and an aqueous gel lubricating and/or wetting agent, wherein the gel comprises methylparaben and/or propylparaben.

In some preferred embodiments, there is provided a urinary catheter kit comprising a container comprising: a urinary catheter comprising a hollow polymeric tubular body comprising a base polymer and an amphiphilic additive; and an aqueous gel lubricating and/or wetting agent, wherein the aqueous gel comprises water in a total amount of between 5-99 wt. %, and wherein the gel comprises methylparaben and/or propylparaben.

In some preferred embodiments, there is provided a urinary catheter kit comprising a container comprising: a urinary catheter comprising a hollow polymeric tubular body comprising a base polymer and an amphiphilic additive; and an aqueous gel lubricating and/or wetting agent, wherein the gel comprises glycerin and at least one alkyl paraben independently selected from the group consisting of: methylparaben, propylparaben, and a combination thereof.

In some preferred embodiments, there is provided a urinary catheter kit comprising a container comprising: a urinary catheter comprising a hollow polymeric tubular body comprising a base polymer and an amphiphilic additive; and an aqueous gel lubricating and/or wetting agent, wherein the aqueous gel comprises water in a total amount of between 5-99 wt. %, and wherein the gel comprises glycerin and at least one alkyl paraben independently selected from the group consisting of: methylparaben, propylparaben, and a combination thereof.

In some preferred embodiments, there is provided a urinary catheter kit comprising a container comprising: a urinary catheter comprising a hollow polymeric tubular body comprising a base polymer and an amphiphilic additive; and an aqueous gel lubricating and/or wetting agent, wherein the gel comprises at least one hydroxyalkylcellulose, preferably comprising hydroxyethylcellulose.

In some preferred embodiments, there is provided a urinary catheter kit comprising a container comprising: a urinary catheter comprising a hollow polymeric tubular body comprising a base polymer and an amphiphilic additive; and an aqueous gel lubricating and/or wetting agent, wherein the aqueous gel comprises water in a total amount of between 5-99 wt. %, and wherein the gel comprises at least one hydroxyalkylcellulose, preferably comprising hydroxyethylcellulose.

In some preferred embodiments, there is provided a urinary catheter kit comprising a container comprising: a urinary catheter comprising a hollow polymeric tubular body comprising a base polymer and an amphiphilic additive; and an aqueous gel lubricating and/or wetting agent, wherein the aqueous gel comprises water in a total amount of between 20-98 wt. %, or between 50-98 wt. %.

According to a fifth aspect of the invention, there is provided a method of lubricating and/or wetting a urinary catheter, the method comprising the steps of:
(a) Providing a urinary catheter comprising a hollow polymeric tubular body comprising a base polymer and an amphiphilic additive; and
(b) Treating the catheter with an aqueous gel.

According to a sixth aspect of the invention, there is provided a method of lubricating and/or wetting a urinary catheter, the method comprising the steps of:
(a) Providing a urinary catheter comprising a hollow polymeric tubular body comprising a base polymer and an amphiphilic additive; and
(b) Treating the catheter with an aqueous gel comprising water in a total amount of between 5-99 wt. %.

The catheter of the fifth and sixth aspects of the invention is preferably the catheter of the first aspect of the invention. The aqueous gel of the fifth aspect of the invention is preferably the aqueous gel of the first aspect of the invention. The aqueous gel of the sixth aspect of the invention is preferably the aqueous gel of the second aspect of the invention.

Statements of invention relating to any other aspect of the present invention may also be applied mutatis mutandis to the fifth and sixth aspects of the invention.

The following statements apply to the fifth and sixth aspects of the invention.

Step (b) preferably comprises treating at least one surface of the catheter with the gel, preferably at least an outer surface thereof.

In some embodiments, step (b) comprises treating at least 20% of the outer surface area of the catheter with the gel, or at least 25, 30, 35, 40, 45, 50, 55, 60, 65, 70, 75, 80, 85, 90, 95, 96, 97, 98, or at least 99% of the outer surface area of the catheter, preferably at least 75%, or at least 90% of the outer surface area, or between 75% and 100% of the outer surface area of the catheter.

In some embodiments, step (b) comprises applying the gel to at least one surface of the catheter, preferably as a layer on the surface. Step (b) may preferably comprise applying the gel to at least part of the outer surface of the catheter.

In some embodiments, step (b) comprises submerging the catheter in the gel.

In some embodiments, step (b) comprises treating the catheter with the gel for a total time of at least 5 seconds, or at least 10, 20, 30, 40, or at least 50 seconds, or at least 1 minute, or at least 2, 3, 4, or at least 5 minutes, or at least 10, 20, 30, 40, or at least 50 minutes, or at least 1 hour, or at least 1.5, 2, 2.5, 3, 3.5, or at least 4 hours. Step (b) may comprise treating the catheter with the gel for a total time of no greater than 1 week, or no greater than 6 days, or no greater than 5, 4, 3, 2, or no greater than 1 day, or no greater than 20 hours, 19, 18, 17, 16, 15, 14, 13, 12, 11, 10, 9, 8, 7, 6, or no greater than 5 hours.

In some embodiments, step (b) is performed at a temperature of at least 1° C., or at least 2, 3, 4, or at least 5° C. In some embodiments, step (b) is performed at a temperature of no greater than 60° C., or no greater than 55, 50, 45, 40, 35, 30, or no greater than 25° C. In some embodiments, step (b) is performed at a temperature of between 1-60° C., or between 5-50, or between 10-40° C.

In some embodiments, step (b) is performed when the catheter is held in a container. The container may preferably be the container of the third aspect of the invention. The container may comprise the gel. Step (b) may comprise placing the catheter into the container to bring the catheter into direct contact with the gel. Step (b) may comprise submerging the catheter in the gel.

In some embodiments, the gel may be contained in a gel container and the catheter may be contained in a separate catheter container, preferably as described for the third aspect of the invention. Step (b) may comprise releasing the gel from the separate gel container, and then treating the catheter with the gel. The gel container may be configured to rupture or break to release the gel upon opening of the catheter container.

In some preferred embodiments, there is provided a method of lubricating and/or wetting a urinary catheter, the method comprising the steps of:
(a) Providing a urinary catheter comprising a hollow polymeric tubular body comprising a base polymer and an amphiphilic additive; and
(b) Treating the catheter with an aqueous gel, wherein the gel has a viscosity of between 5000-800000 cP.

In some preferred embodiments, there is provided a method of lubricating and/or wetting a urinary catheter, the method comprising the steps of:
(a) Providing a urinary catheter comprising a hollow polymeric tubular body comprising a base polymer and an amphiphilic additive; and
(b) Treating the catheter with an aqueous gel comprising water in a total amount of between 5-99 wt. %, wherein the gel has a viscosity of between 5000-800000 cP.

In some preferred embodiments, there is provided a method of lubricating and/or wetting a urinary catheter, the method comprising the steps of:
(a) Providing a urinary catheter comprising a hollow polymeric tubular body comprising a base polymer and an amphiphilic additive; and
(b) Treating the catheter with an aqueous gel, wherein the gel has a pH of between 4-7.

In some preferred embodiments, there is provided a method of lubricating and/or wetting a urinary catheter, the method comprising the steps of:
(a) Providing a urinary catheter comprising a hollow polymeric tubular body comprising a base polymer and an amphiphilic additive; and
(b) Treating the catheter with an aqueous gel comprising water in a total amount of between 5-99 wt. %, wherein the gel has a pH of between 4-7.

In some preferred embodiments, there is provided a method of lubricating and/or wetting a urinary catheter, the method comprising the steps of:
(a) Providing a urinary catheter comprising a hollow polymeric tubular body comprising a base polymer and an amphiphilic additive; and
(b) Treating the catheter with an aqueous gel, wherein the gel comprises at least one paraben.

In some preferred embodiments, there is provided a method of lubricating and/or wetting a urinary catheter, the method comprising the steps of:
(a) Providing a urinary catheter comprising a hollow polymeric tubular body comprising a base polymer and an amphiphilic additive; and
(b) Treating the catheter with an aqueous gel comprising water in a total amount of between 5-99 wt. %, wherein the gel comprises at least one paraben.

In some preferred embodiments, there is provided a method of lubricating and/or wetting a urinary catheter, the method comprising the steps of:
(a) Providing a urinary catheter comprising a hollow polymeric tubular body comprising a base polymer and an amphiphilic additive; and
(b) Treating the catheter with an aqueous gel, wherein the gel comprises glycerin and at least one paraben.

In some preferred embodiments, there is provided a method of lubricating and/or wetting a urinary catheter, the method comprising the steps of:
(a) Providing a urinary catheter comprising a hollow polymeric tubular body comprising a base polymer and an amphiphilic additive; and
(b) Treating the catheter with an aqueous gel comprising water in a total amount of between 5-99 wt. %, wherein the gel comprises glycerin and at least one paraben.

In some preferred embodiments, there is provided a method of lubricating and/or wetting a urinary catheter, the method comprising the steps of:
(a) Providing a urinary catheter comprising a hollow polymeric tubular body comprising a base polymer and an amphiphilic additive; and
(b) Treating the catheter with an aqueous gel, wherein the gel comprises at least one paraben in a total amount of between 0.05-0.3 wt. %.

In some preferred embodiments, there is provided a method of lubricating and/or wetting a urinary catheter, the method comprising the steps of:
(a) Providing a urinary catheter comprising a hollow polymeric tubular body comprising a base polymer and an amphiphilic additive; and
(b) Treating the catheter with an aqueous gel comprising water in a total amount of between 5-99 wt. %, wherein the gel comprises at least one paraben in a total amount of between 0.05-0.3 wt. %.

In some preferred embodiments, there is provided a method of lubricating and/or wetting a urinary catheter, the method comprising the steps of:
(a) Providing a urinary catheter comprising a hollow polymeric tubular body comprising a base polymer and an amphiphilic additive; and
(b) Treating the catheter with an aqueous gel, wherein the gel comprises at least one paraben comprising at least one alkyl paraben, preferably at least one C₁-C₁₀ alkyl paraben.

In some preferred embodiments, there is provided a method of lubricating and/or wetting a urinary catheter, the method comprising the steps of:
(a) Providing a urinary catheter comprising a hollow polymeric tubular body comprising a base polymer and an amphiphilic additive; and
(b) Treating the catheter with an aqueous gel comprising water in a total amount of between 5-99 wt. %, wherein the gel comprises at least one paraben comprising at least one alkyl paraben, preferably at least one C₁-C₁₀ alkyl paraben.

In some preferred embodiments, there is provided a method of lubricating and/or wetting a urinary catheter, the method comprising the steps of:
(a) Providing a urinary catheter comprising a hollow polymeric tubular body comprising a base polymer and an amphiphilic additive; and
(b) Treating the catheter with an aqueous gel, wherein the gel comprises at least one paraben comprising at least one alkyl paraben that is independently selected from the group consisting of: methylparaben, ethylparaben, propylparaben, butylparaben, and combinations thereof.

In some preferred embodiments, there is provided a method of lubricating and/or wetting a urinary catheter, the method comprising the steps of:
(a) Providing a urinary catheter comprising a hollow polymeric tubular body comprising a base polymer and an amphiphilic additive; and
(b) Treating the catheter with an aqueous gel comprising water in a total amount of between 5-99 wt. %, wherein the gel comprises at least one paraben comprising at least one alkyl paraben that is independently selected from the group consisting of: methylparaben, ethylparaben, propylparaben, butylparaben, and combinations thereof.

In some preferred embodiments, there is provided a method of lubricating and/or wetting a urinary catheter, the method comprising the steps of:
(a) Providing a urinary catheter comprising a hollow polymeric tubular body comprising a base polymer and an amphiphilic additive; and
(b) Treating the catheter with an aqueous gel, wherein the gel comprises methylparaben and/or propylparaben.

In some preferred embodiments, there is provided a method of lubricating and/or wetting a urinary catheter, the method comprising the steps of:
(a) Providing a urinary catheter comprising a hollow polymeric tubular body comprising a base polymer and an amphiphilic additive; and
(b) Treating the catheter with an aqueous gel comprising water in a total amount of between 5-99 wt. %, wherein the gel comprises methylparaben and/or propylparaben.

In some preferred embodiments, there is provided a method of lubricating and/or wetting a urinary catheter, the method comprising the steps of:
(a) Providing a urinary catheter comprising a hollow polymeric tubular body comprising a base polymer and an amphiphilic additive; and
(b) Treating the catheter with an aqueous gel, wherein the gel comprises glycerin and at least one alkyl paraben independently selected from the group consisting of: methylparaben, propylparaben, and a combination thereof.

In some preferred embodiments, there is provided a method of lubricating and/or wetting a urinary catheter, the method comprising the steps of:
(a) Providing a urinary catheter comprising a hollow polymeric tubular body comprising a base polymer and an amphiphilic additive; and
(b) Treating the catheter with an aqueous gel comprising water in a total amount of between 5-99 wt. %, wherein the gel comprises glycerin and at least one alkyl paraben independently selected from the group consisting of: methylparaben, propylparaben, and a combination thereof.

In some preferred embodiments, there is provided a method of lubricating and/or wetting a urinary catheter, the method comprising the steps of:
(a) Providing a urinary catheter comprising a hollow polymeric tubular body comprising a base polymer and an amphiphilic additive; and
(b) Treating the catheter with an aqueous gel, wherein the gel comprises at least one hydroxyalkylcellulose, preferably comprising hydroxyethylcellulose.

In some preferred embodiments, there is provided a method of lubricating and/or wetting a urinary catheter, the method comprising the steps of:
(a) Providing a urinary catheter comprising a hollow polymeric tubular body comprising a base polymer and an amphiphilic additive; and
(b) Treating the catheter with an aqueous gel comprising water in a total amount of between 5-99 wt. %, wherein the gel comprises at least one hydroxyalkylcellulose, preferably comprising hydroxyethylcellulose.

In some preferred embodiments, there is provided a method of lubricating and/or wetting a urinary catheter, the method comprising the steps of:
(a) Providing a urinary catheter comprising a hollow polymeric tubular body comprising a base polymer and an amphiphilic additive; and
(b) Treating the catheter with an aqueous gel comprising water in a total amount of between 20-98 wt. %, or between 50-98 wt. %.

According to a seventh aspect of the invention, there is provided the use of an aqueous gel as a lubricating and/or wetting agent for a urinary catheter, the catheter comprising a hollow polymeric tubular body comprising a base polymer and an amphiphilic additive.

According to an eighth aspect of the invention, there is provided the use of an aqueous gel as a lubricating and/or wetting agent for a urinary catheter, the catheter comprising a hollow polymeric tubular body comprising a base polymer and an amphiphilic additive, and wherein the gel comprises water in a total amount of between 5-99 wt. %.

The catheter of the seventh and eighth aspects of the invention is preferably the catheter of the first aspect of the invention. The aqueous gel of the seventh aspect of the invention is preferably the aqueous gel of the first aspect of the invention. The aqueous gel of the eighth aspect of the invention is preferably the aqueous gel of the second aspect of the invention.

Statements of invention relating to any other aspect of the present invention may also be applied mutatis mutandis to the seventh and eighth aspects of the invention.

The following statements apply to the seventh and eighth aspects of the invention.

In some embodiments, the use comprises treating the catheter with the gel, preferably at least an outer surface thereof.

In some embodiments, the catheter may be used immediately after treatment with the gel.

In some preferred embodiments, there is provided the use of an aqueous gel as a lubricating and/or wetting agent for a urinary catheter, the catheter comprising a hollow polymeric tubular body comprising a base polymer and an amphiphilic additive, wherein the gel has a viscosity of between 5000-800000 cP.

In some preferred embodiments, there is provided the use of an aqueous gel as a lubricating and/or wetting agent for a urinary catheter, the catheter comprising a hollow polymeric tubular body comprising a base polymer and an amphiphilic additive, and wherein the gel comprises water in a total amount of between 5-99 wt. %, and wherein the gel has a viscosity of between 5000-800000 cP.

In some preferred embodiments, there is provided the use of an aqueous gel as a lubricating and/or wetting agent for a urinary catheter, the catheter comprising a hollow polymeric tubular body comprising a base polymer and an amphiphilic additive, wherein the gel has a pH of between 4-7.

In some preferred embodiments, there is provided the use of an aqueous gel as a lubricating and/or wetting agent for a urinary catheter, the catheter comprising a hollow polymeric tubular body comprising a base polymer and an amphiphilic additive, and wherein the gel comprises water in a total amount of between 5-99 wt. %, and wherein the gel has a pH of between 4-7.

In some preferred embodiments, there is provided the use of an aqueous gel as a lubricating and/or wetting agent for a urinary catheter, the catheter comprising a hollow polymeric tubular body comprising a base polymer and an amphiphilic additive, wherein the gel comprises at least one paraben.

In some preferred embodiments, there is provided the use of an aqueous gel as a lubricating and/or wetting agent for a urinary catheter, the catheter comprising a hollow polymeric tubular body comprising a base polymer and an amphiphilic additive, and wherein the gel comprises water in a total amount of between 5-99 wt. %, and wherein the gel comprises at least one paraben.

In some preferred embodiments, there is provided the use of an aqueous gel as a lubricating and/or wetting agent for a urinary catheter, the catheter comprising a hollow polymeric tubular body comprising a base polymer and an amphiphilic additive, wherein the gel comprises glycerin and at least one paraben.

In some preferred embodiments, there is provided the use of an aqueous gel as a lubricating and/or wetting agent for a urinary catheter, the catheter comprising a hollow polymeric tubular body comprising a base polymer and an amphiphilic additive, and wherein the gel comprises water in a total amount of between 5-99 wt. %, and wherein the gel comprises glycerin and at least one paraben.

In some preferred embodiments, there is provided the use of an aqueous gel as a lubricating and/or wetting agent for a urinary catheter, the catheter comprising a hollow polymeric tubular body comprising a base polymer and an amphiphilic additive, wherein the gel comprises at least one paraben in a total amount of between 0.05-0.3 wt. %.

In some preferred embodiments, there is provided the use of an aqueous gel as a lubricating and/or wetting agent for a urinary catheter, the catheter comprising a hollow polymeric tubular body comprising a base polymer and an amphiphilic additive, and wherein the gel comprises water in a total amount of between 5-99 wt. %, and wherein the gel comprises at least one paraben in a total amount of between 0.05-0.3 wt. %.

In some preferred embodiments, there is provided the use of an aqueous gel as a lubricating and/or wetting agent for a urinary catheter, the catheter comprising a hollow polymeric tubular body comprising a base polymer and an amphiphilic additive, wherein the gel comprises at least one paraben comprising at least one alkyl paraben, preferably at least one C₁-C₁₀ alkyl paraben.

In some preferred embodiments, there is provided the use of an aqueous gel as a lubricating and/or wetting agent for a urinary catheter, the catheter comprising a hollow polymeric tubular body comprising a base polymer and an amphiphilic additive, and wherein the gel comprises water in a total amount of between 5-99 wt. %, and wherein the gel comprises at least one paraben comprising at least one alkyl paraben, preferably at least one C₁-C₁₀ alkyl paraben.

In some preferred embodiments, there is provided the use of an aqueous gel as a lubricating and/or wetting agent for a urinary catheter, the catheter comprising a hollow polymeric tubular body comprising a base polymer and an amphiphilic additive, wherein the gel comprises at least one paraben comprising at least one alkyl paraben that is independently selected from the group consisting of: methylparaben, ethylparaben, propylparaben, butylparaben, and combinations thereof.

In some preferred embodiments, there is provided the use of an aqueous gel as a lubricating and/or wetting agent for a urinary catheter, the catheter comprising a hollow polymeric tubular body comprising a base polymer and an amphiphilic additive, and wherein the gel comprises water in a total amount of between 5-99 wt. %, and wherein the gel comprises at least one paraben comprising at least one alkyl paraben that is independently selected from the group consisting of: methylparaben, ethylparaben, propylparaben, butylparaben, and combinations thereof.

In some preferred embodiments, there is provided the use of an aqueous gel as a lubricating and/or wetting agent for a urinary catheter, the catheter comprising a hollow polymeric tubular body comprising a base polymer and an amphiphilic additive, wherein the gel comprises methylparaben and/or propylparaben.

In some preferred embodiments, there is provided the use of an aqueous gel as a lubricating and/or wetting agent for a urinary catheter, the catheter comprising a hollow polymeric tubular body comprising a base polymer and an amphiphilic additive, and wherein the gel comprises water in a total amount of between 5-99 wt. %, and wherein the gel comprises methylparaben and/or propylparaben.

In some preferred embodiments, there is provided the use of an aqueous gel as a lubricating and/or wetting agent for a urinary catheter, the catheter comprising a hollow polymeric tubular body comprising a base polymer and an amphiphilic additive, wherein the gel comprises glycerin and at least one alkyl paraben independently selected from the group consisting of: methylparaben, propylparaben, and a combination thereof.

In some preferred embodiments, there is provided the use of an aqueous gel as a lubricating and/or wetting agent for a urinary catheter, the catheter comprising a hollow polymeric tubular body comprising a base polymer and an amphiphilic additive, and wherein the gel comprises water in a total amount of between 5-99 wt. %, and wherein the gel comprises glycerin and at least one alkyl paraben independently selected from the group consisting of: methylparaben, propylparaben, and a combination thereof.

In some preferred embodiments, there is provided the use of an aqueous gel as a lubricating and/or wetting agent for a urinary catheter, the catheter comprising a hollow polymeric tubular body comprising a base polymer and an amphiphilic additive, wherein the gel comprises at least one hydroxyalkylcellulose, preferably comprising hydroxyethylcellulose.

In some preferred embodiments, there is provided the use of an aqueous gel as a lubricating and/or wetting agent for a urinary catheter, the catheter comprising a hollow polymeric tubular body comprising a base polymer and an amphiphilic additive, and wherein the gel comprises water in a total amount of between 5-99 wt. %, and wherein the gel comprises at least one hydroxyalkylcellulose, preferably comprising hydroxyethylcellulose.

In some preferred embodiments, there is provided the use of an aqueous gel as a lubricating and/or wetting agent for a urinary catheter, the catheter comprising a hollow polymeric tubular body comprising a base polymer and an amphiphilic additive, and wherein the gel comprises water in a total amount of between 20-98 wt. %, or between 50-98 wt. %.

### DETAILED DESCRIPTION OF THE INVENTION

In order that the invention may be more clearly understood, embodiments thereof will now be described, by way of example only:

### Urinary Catheter Kit

A urinary catheter kit was provided. The kit comprises a container in the form of a pouch. The pouch contained an intermittent urinary catheter comprising a hollow polymeric tubular body comprising a base polymer formed from a thermoplastic elastomeric material and further comprising an amphiphilic additive. The container was filled with an aqueous gel comprising water, glycerin, methyl paraben, and propyl paraben. The intermittent catheter was submerged in the aqueous gel.

The catheter could simply be removed from the container and used in the conventional manner, without any requirement to rinse the catheter after removal from the gel and before use. On removal from the container, the outer surface of the catheter body is coated with a layer of the gel. Catheter surface lubricity was excellent, and the catheter could be inserted and removed easily with minimal discomfort caused.

The use of aqueous gel wetting and/or lubricating agents with urinary catheters comprising amphiphilic additives is evaluated in the laboratory tests described below.

### Example 1—Coefficient of Friction Testing (Catheter with Amphiphilic Additive Vs Catheter without Additive)

### Gels Tested

Three different aqueous gels were provided for the investigation (Gel 1, Gel 2, and Gel 3).

The composition and viscosity and pH values of the three gels are displayed in Table 1 below.

TABLE 1

| | Viscosity | | Methylparaben | Propylparaben | Glycerin | Water |
|---|---|---|---|---|---|---|
| Gel | 1000 cP | pH | (wt. %) | (wt. %) | (wt. %) | (wt. %) |
| 1 | 18-26.5 | 5.0-6.0 | 0.07-0.10 | 0.02-0.03 | 31.5-38.5 | 56.9-69.6 |
| 2 | 13-18 | 5.0-6.0 | 0.15-0.22 | 0.04-0.06 | 60.3-73.7 | 28.0-36.0 |
| 3 | 300 | 5.2 | 0.18 | 0.02 | 9.0-11.0 | 77.8-95.0 |

### Catheters Tested

The Following Catheters were Tested:
Catheter 1 (for inventive runs)—a urinary catheter comprising a hollow polymeric tubular body comprising a base polymer formed from a thermoplastic elastomeric material and further comprising an amphiphilic additive.
Catheter 2 (control)—a urinary catheter comprising a hollow polymeric tubular body comprising a base polymer formed of PVC. No additive.

### Sample Preparation

Catheters were stored independently stored in the respective gels for one hour prior to testing. Catheters were then removed from the gel and the gel cleaned out. 2 mL of the test gel was then added to the surface of the catheter body before testing.

Catheters were then tested to determine coefficient of friction.

### Results

Mean coefficient of friction values are summarised in Tables 2 (Catheter 1) and 3 (Catheter 2) below.

TABLE 2
Coefficient of Friction (COF)-Catheter 1 (Inventive Runs)

| Gel | 1 | 2 | 3 |
|---|---|---|---|
| Mean COF | 0.0979 | 0.0636 | 0.0444 |
| SD | 0.0070944 | 0.0051159 | 0.0036405 |
| % RSD | 7.2451 | 8.0465 | 8.1994 |

TABLE 3
Coefficient of Friction (COF)-Catheter 2 (Control Runs)

| Gel | 1 | 2 | 3 |
|---|---|---|---|
| Mean COF | 0.28396 | 0.2082 | 0.39024 |
| SD | 0.0718951 | 0.0336550 | 0.0478298 |
| % RSD | 25.3187 | 16.1648 | 12.2565 |

From a review of the results, when analysed using the same test conditions, coefficient of friction results observed for gel-lubricated Catheter 1 (inventive—amphiphilic additive) are significantly improved compared to gel-lubricated Catheter 2 (control—no additive). It was further noted that the inventive gel-lubricated catheters felt more slippery and lubricated than the control gel-lubricated catheters.

Coefficient of friction results achieved for the inventive gel-lubricated catheters were also surprisingly in line with values for the same catheter (Catheter 1) that was wetted with water instead of lubricated with a gel, despite the presence of viscous gels on the catheter surface.

### Gel Performance

The three different gels tested provided differing results for the inventive runs performed using Catheter 1. As shown above, gels with higher water contents were found to provide the most optimal coefficients of friction compared to those with lower water contents.

The surface chemistry created by the amphiphilic additive in Catheter 1 is hydrophilic in nature. Without wishing to be bound by theory, gels with a higher water content allow for improved interactions between the gel and amphiphilic additive molecules of the catheter, in particular hydrophilic portions of the amphiphilic additive molecules. A hydration layer is created through temporary hydrogen bonds on the catheter surface between the additive molecules and water molecules present in the gel. As such, a greater number of amphiphilic additive molecules (especially hydrophilic portions thereof) are present at or on an outer surface of the catheter, which provides for excellent and long-lasting catheter lubricity.

### Example 2—Coefficient of Friction Testing Using Further Gels (Catheter with Amphiphilic Additive Vs Catheter without Additive)

To further analyse the compatibility between amphiphilic additive containing catheters and gels, a range of additional gels were tested.

### Further Gels Tested

The following further gels were tested.

Gel 4—hydroxyethylcellulose (1-3 wt. %); glycerin (10-25 wt. %); citric acid (0.01-0.1 wt. %); propylparaben (0.05-0.2 wt. %); methylparaben (0.05-0.2 wt. %); and deionised water (q.s 100 wt. %).

Gel 5—glycerin; polyethyleneglycol-300; polyethyleneglycol-1450; sodium hydroxide; methylparaben; propylparaben; purified water.

Gel 6—lidocaine hydrochloride (2 wt. %); glycerin; hydrochloric acid; hydroxyethylcellulose; sodium hydroxide; water.

Gel 7—polyhexamethylene biguanide hydrochloride (0.1-<0.25 wt. %); glycerin; hydroxyethylcellulose.

### Catheters Tested

The catheters tested were the same as for Example 1 above.

### Sample Preparation

Sample preparation was performed as for Example 1 above.

### Results

Mean coefficient of friction values are summarised in Tables 4 (Catheter 1) and 5 (Catheter 2) below.

TABLE 4
Coefficient of Friction (COF)-Catheter 1 (Inventive Runs)

| Gel | 4 | 5 | 6 | 7 |
|---|---|---|---|---|
| Mean COF | 0.0504 | 0.0506 | 0.0421 | 0.0378 |
| SD | 0.0054373 | 0.0051129 | 0.0070193 | 0.0078144 |
| % RSD | 10.7883 | 10.1046 | 16.6728 | 20.6512 |

TABLE 5
Coefficient of Friction (COF)-Catheter 2 (Control Runs)

| Gel | 4 | 5 | 6 | 7 |
|---|---|---|---|---|
| Mean COF | 0.1915 | 0.3180 | 0.1938 | 0.2049 |
| SD | 0.0351385 | 0.0937929 | 0.0445635 | 0.0423899 |
| % RSD | 18.35 | 29.49 | 22.99 | 20.69 |

The trend observed was similar to Example 1, with coefficient of friction results observed for gel-lubricated Catheter 1 (inventive—amphiphilic additive) being significantly improved compared to gel-lubricated Catheter 2 (control—no additive).

Coefficient of friction results achieved for the inventive gel-lubricated catheters were again surprisingly in line with values for the same catheter (Catheter 1) that was wetted with water instead of lubricated with a gel, despite the presence of viscous gels on the catheter surface.

### Example 3—Coefficient of Friction Testing (Catheter with Amphiphilic Additive Vs Catheter with Hydrophilic Additive)

To compare the coefficient of friction performance when coated with gels of catheters containing amphiphilic additives with catheters containing hydrophilic additives, the following test was performed.

### Gels Tested

An aqueous gel, Gel 8, was tested.

The composition and viscosity and pH values of the gel is displayed in Table 6 below.

TABLE 6

| | | | Methyl- | Propyl- | | |
|---|---|---|---|---|---|---|
| | Viscosity | | paraben | paraben | Glycerin | Water |
| Gel | 1000 cP | pH | (wt. %) | (wt. %) | (wt. %) | (wt. %) |
| 8 | 200-400 | 5.0-6.0 | 0.18 | 0.05 | 60.3-73.7 | 28.0-36.0 |

### Catheter Tested

Catheter 3 (non-inventive runs)—a urinary catheter comprising a hollow polymeric tubular body comprising a base polymer and further comprising a hydrophilic polyvinylpyrrolidone (PVP) additive.

### Sample Preparation

Sample preparation was performed as for Example 1 above.

### Results

Mean coefficient of friction values are summarised in Table 7 below.

TABLE 4
Coefficient of Friction (COF) - Catheter 3 (Non-inventive Runs)

| | |
|---|---|
| Gel | 8 |
| Mean COF | 0.1447 |
| SD | 0.0149693 |
| % RSD | 10.35 |

Coefficient of friction values for the hydrophilic additive-containing catheter were considerably higher and worse than for all inventive runs performed with the amphiphilic additive-containing catheter.

This was somewhat surprising given that both catheters comprising amphiphilic additives and catheters comprising hydrophilic additives result in catheters with a hydrophilic surface.

Without wishing to be bound by theory, the discrepancy between these two catheter types may be due to gels hindering absorption of a sufficient volume of water by the hydrophilic additive-containing catheter to become fully lubricated.

This highlights a key advantage of using an amphiphilic additive containing-catheter with a gel lubricant, as provided by the present invention, as the catheter is able to become highly lubricious immediately upon contact with the aqueous gel.

### Example 4—Gel Migration Testing

The following test was performed to evaluate the effectiveness of gel lubrication following catheter insertion.

### Catheters Tested

The catheters tested were the same as for Example 1 above.

### Gels Tested

The gels tested were the same as for Example 1 above. However, a red dye was added to the gels to give them a red colouration and allow for their visualisation in agar gel channels.

### Sample Preparation

Sample preparation was performed as for Example 1 above.

### Test

Agar gel channels with entrance and exit holes on either end were provided to model a catheter being passed through the body.

The catheters coated with the red-dyed gels were independently inserted in through the entrance hole of the agar gel channel, pushed through, and then removed from the exit hole.

Following removal of the catheters, agar gel channels were cut in half and the insides of the channels observed to determine migration of the coating gel through the channels.

### Results

For all gel-coated Catheter 1 runs (inventive—amphiphilic additive), excellent gel migration was observed, with red colouration observable across the entire length of the channel.

This shows that the gel lubricant is available to lubricate the amphiphilic additive-containing catheter along the complete length of the channel.

These results contrasted with those achieved for gel-coated Catheter 2 runs (control—no additive). In these runs, the lubricating gel was found concentrated towards the opening of the channel, but very little to no gel was found further into the channel. This shows that as the catheter moves through the channel, the gel coating is easily lost and less gel is available to lubricate the catheter.

This again highlights the superiority and advantages of using an amphiphilic additive containing-catheter with a gel lubricant, as provided by the present invention.

The above embodiments are described by way of example only. Many variations are possible without departing from the scope of the invention as defined in the appended claims.

1. A urinary catheter comprising a hollow polymeric tubular body comprising a base polymer and an amphiphilic additive, wherein at least a part of the body is coated with an aqueous gel, wherein the gel comprises water in a total amount of between 5-99 wt. %.

2. A urinary catheter as claimed in claim 1, wherein the catheter is an intermittent catheter.

3. A urinary catheter as claimed in any preceding claim, wherein the amphiphilic additive is polymeric or oligomeric.

4. A urinary catheter as claimed in any preceding claim, wherein the amphiphilic additive is an amphiphilic A-B block copolymer comprising a hydrophobic hydrocarbon A-block and a hydrophilic B-block.

5. A urinary catheter as claimed in claim 4, wherein the A-block of the additive comprises a hydrocarbon chain block of the formula CH₃CH₂ (CH₂CH₂)ₐ where “a” is 5-25 and preferably 9-25.

6. A urinary catheter as claimed in claim 4 or 5, wherein the B-block is a hydrophilic oligomer comprising between 2 and 10 monomer units optionally derived from monomers chosen from: alkylene oxides, alkylene glycols, epihalohydrins, unsaturated carboxylic acids, alkylene imines, lactones, vinyl alcohol, and vinyl alkanoates.

7. A urinary catheter as claimed in any preceding claim, wherein the base polymer is independently chosen from: polyolefins, polyesters, polyacrylates, polyamides, thermoplastic elastomeric material, polyether block amide, thermoplastic vulcanizates, thermoplastic copolyesters, thermoplastic polyamides, fluororubber, water disintegrable or enzymatically hydrolysable material, and combinations, blends or copolymers of any of the above materials, and wherein the base polymer preferably comprises a polymer independently chosen from: polyolefins, polyvinyl chloride, polyurethane, styrene-butadiene copolymer (SBC), styrene-ethylene-butylene-styrene copolymer (SEBS), thermoplastic elastomeric material, and combinations, blends or copolymers of any of the above materials.

8. A urinary catheter as claimed in any preceding claim, wherein an outer surface of the body is coated with the aqueous gel, preferably at least 50% of the outer surface area of the body.

9. A urinary catheter as claimed in any preceding claim, wherein the aqueous gel is present as a layer on at least part of the body.

10. A urinary catheter as claimed in any preceding claim, wherein the gel has a viscosity of between 5000-800000 cP.

11. A urinary catheter as claimed in any preceding claim, wherein the gel has a pH of between 4-7.

12. A urinary catheter as claimed in any preceding claim, wherein the gel comprises water in a total amount of between 20-98 wt. %, preferably between 50-98 wt. %.

13. A urinary catheter as claimed in any preceding claim, wherein the gel comprises glycerin and/or at least one paraben.

14. A urinary catheter as claimed in claim 13, wherein the gel comprises glycerin in a total amount of between 5-80 wt. %.

15. A urinary catheter as claimed in claim 13 or 14, wherein the gel comprises the at least one paraben in a total amount of between 0.05-0.3 wt. %.

16. A urinary catheter as claimed in any one of claims 13 to 15, wherein the at least one paraben comprises at least one alkyl paraben, preferably at least one C₁-C₁₀ alkyl paraben.

17. A urinary catheter as claimed in claim 16, wherein the at least one alkyl paraben is independently chosen from: methylparaben, ethylparaben, propylparaben, butylparaben, and combinations thereof.

18. A urinary catheter as claimed in claim 17, wherein the at least one alkyl paraben comprises methylparaben and/or propylparaben.

19. A urinary catheter as claimed in 18, wherein the gel comprises glycerin and at least one alkyl paraben independently chosen from: methylparaben, propylparaben, and a combination thereof.

20. A urinary catheter as claimed in any preceding claim, wherein the gel comprises at least one hydroxyalkylcellulose, preferably comprising hydroxyethylcellulose.

21. A urinary catheter as claimed in any preceding claim, wherein the aqueous gel comprises at least one species that is independently chosen from: a chlorine-containing species, a peroxide species, a base, an acid, a photosensitiser, a permanganate species, an alcohol, a phenol, an aldehyde, ionic silver, molecular iodine or an iodophor, an imine-containing species, a salt and combinations thereof, and wherein the aqueous gel preferably comprises at least one chlorine-containing species independently chosen from: hypochlorous acid, at least one hypochlorite salt, chlorine dioxide, molecular chlorine, a dichloroisocyanurate salt, chloroazodin, dichlorodimethylhydantoin, chloroxylenol, chlorhexidine, and combinations thereof.

22. A urinary catheter kit comprising a container comprising: a urinary catheter comprising a hollow polymeric tubular body comprising a base polymer and an amphiphilic additive; and an aqueous gel lubricating and/or wetting agent, wherein the aqueous gel comprises water in a total amount of between 5-99 wt. %.

23. A urinary catheter kit as claimed in claim 22, wherein the aqueous gel is in direct contact with the catheter, preferably with at least one surface thereof, and wherein the catheter is optionally submerged in the aqueous gel.

24. A urinary catheter kit as claimed in claim 22, wherein the aqueous gel is contained in a separate gel container that is located in the container and the gel is not in direct contact with the catheter.

25. A method of lubricating and/or wetting a urinary catheter, the method comprising the steps of:
a. Providing a urinary catheter comprising a hollow polymeric tubular body comprising a base polymer and an amphiphilic additive; and
b. Treating the catheter with an aqueous gel comprising water in a total amount of between 5-99 wt. %.

26. A method as claimed in claim 25, wherein step (b) comprises treating at least one surface of the catheter with the gel, preferably at least an outer surface of the catheter.

27. Use of an aqueous gel as a lubricating and/or wetting agent for a urinary catheter, the catheter comprising a hollow polymeric tubular body comprising a base polymer and an amphiphilic additive, and wherein the gel comprises water in a total amount of between 5-99 wt. %.
